(19) United States
(12) Patent Application Publication
(10) Pub. No.: US 2010/0186357 A1
(43) Pub. Date: Jul. 29, 2010

(54) AIR CLEANER

(76) Inventors: Yasukata Takeda, Osaka (JP); Masaki Ohtsuka, Osaka (JP)

Correspondence Address: BIRCH STEWART KOLASCH & BIRCH, PO BOX 747, FALLS CHURCH, VA, 22040-0747, US

(21) Appl. No.: 12/667,841

(22) PCT Filed: Nov. 2, 2007
(86) PCT No.: PCT/JP2007/071370

§ 371 Date: Jan. 5, 2010

(30) Foreign Application Priority Data
Jul. 30, 2007 (JP) 2007-197567

(51) Int. Cl. B01D 46/00; F24F 7/00; A61L 9/00

(52) U.S. Cl. 55/471

(57) ABSTRACT

Provided is an air cleaner capable of enhancing an effect of improving an indoor air environment through enhancing an efficiency of circulating an air current without increasing an air flow volume. The air cleaner (1) is placed on a floor surface (F) and used so as to be located in the vicinity of a side wall surface (W1) of a room and comprises a suction port (122), an air filter (150), a blowout port (131), and an air blower (170). When a distance from a middle position of the blowout port (131) to the side wall surface (W1) of the room is supposed to be L [mm], a distance from the middle position of the blowout port (131) to a ceiling surface of the room is supposed to be H [mm], and the air cleaner (1) is placed and used in a position which allows the distance L to be a value selected from among values in a range of 100<L<600, in order to cause the air sent out from the blowout port (131) to first reach the side wall surface (W1) of the room, an angle θ[°] formed between a direction in which the air is sent out from the blowout port (131) and a vertically upward direction is set to be in a range of tan⁻¹(L/H)<θ≦35.

### TECHNICAL FIELD

The present invention relates generally to air cleaners, and, more particularly, to an air cleaner which is placed on a floor or a desk and used so as to be located in the vicinity of a sidewall surface of a room.

### BACKGROUND ART

In recent years, since houses have been rendered highly airtight, pollutants are easily trapped in rooms. This is posing serious problems such as a sick house syndrome caused by volatile organic compounds and allergic symptoms caused by house dust. Therefore, in order to improve an indoor air environment, an air cleaner and an air conditioner with an air cleaning function are used. In general, as a method for cleaning indoor air, a method in which pollutants are taken into an air cleaner by an air blower and removed by a filter is employed. On the other hand, in order to improve the indoor air environment, active researches have been conducted, devising a method in which instead of the filter, an electric dust collecting apparatus is used, a method in which ions or ozone is used, or the like.

Furthermore, a method in which the indoor air environment is improved by an air current sent out from a blowout port of an air cleaner has been proposed.

For example, in Japanese Patent Application Laid-Open Publication No. 2006-17343 (Patent Document 1), an air cleaner capable of quickly cleaning up a room by supplying cleaned air both in a lateral direction and a forward direction has been proposed.

In addition, in Japanese Patent Application Laid-Open Publication No. 2006-57919 (Patent Document 2), an air cleaner capable of quickly searching air pollution in a room by detecting dust or odors with a sensor while a movable louver provided in a blowout port is being automatically moved back and forth or right and left to change an air current in a room has been proposed.

Besides the air cleaners disclosed in the above-cited documents, a variety of air cleaners with many various ideas devised have been proposed.

For example, FIG. 34 and FIG. 35 are side sectional views, each of which schematically illustrates one example of a conventional air cleaner.

As shown in FIG. 34, a main body of the conventional air cleaner 10 is held by a housing 1000. The housing 1000 is placed on a floor surface F and has a front surface (anterior surface) 1100, a rear surface (posterior surface) 1200, a top surface (upper surface) 1300, and a bottom surface 1400 contacting the floor surface F. A side of the front surface 1100 of the housing 1000 is covered by a panel 1110 and a suction port 1120 is provided in a position facing the panel 1110. The housing 1000 is placed on the floor surface F so as to allow a side of the rear surface 1200 to face a side wall surface W1 of a room and to provide a spacing of approximately 300 mm from the side wall surface W1. On the top surface 1300 of the housing 1000, a blowout port 1310 is provided. The blowout port 1310 is formed so as to be of a substantially rectangular shape extending in a width direction of the housing 1000 and is provided so as to face vertically upward. Inside of the housing 1000, an air flow channel which communicates from the suction port 1120 to the blowout port 1310 and includes a suction flow passageway 1600 and a blowout flow passageway 1800 is formed. In the air flow channel, an air blower 1700 for sending out air is placed. As a fan constituting the air blower 1700, a sirocco fan 1710 is used. In a position facing the panel 1110, an air filter 1500 for collecting and removing dust and/or odors (pollutants) which are present in the air sucked from the suction port 1120 is provided.

In a conventional air cleaner 20 shown in FIG. 35, an operation part 1900 is provided on a top surface 1300 of a housing 1000 and a control board 1920 is provided under the operation part 1900 and inside the housing 1000. In addition, in a lower portion of a side of a rear surface 1200 of the housing 1000, a power source board 1930 is provided under an air blower 1700.

Upon starting an operation of the air cleaner having the above-described configuration, as shown in FIG. 34, the sirocco fan 1710 is driven in a rotated manner and the air are sucked from the suction port 1120 in a direction indicated by an arrow U and the dust and/or odors (pollutants) which are present in the air is removed by the air filter 1500. The air which has taken into the suction flow passageway 1600 in the housing 1000 and from which the dust has been removed by the air filter 1500 flows through the blowout flow passageway 1800 and is sent out into the room from the blowout port 1310 vertically upward in a direction indicated by an arrow V. The air sent out into the room reaches a ceiling surface of the room, circulates in the room, and is sucked into the suction port 1120 of the air cleaner 10.

Patent Document 1: Japanese Patent Application Laid-Open Publication No. 2006-17343

Patent Document 2: Japanese Patent Application Laid-Open Publication No. 2006-57919

### DISCLOSURE OF THE INVENTION

### Problems to be Solved by the Invention

In recent years, solving a problem of air pollution in a room has been strongly demanded and there has been required to further improve an indoor air environment by using an air cleaner. In order to improve the indoor air environment by using the air cleaner, besides the technologies proposed in the above-mentioned documents, a variety of technologies have been studied.

However, since the conventional method for quickly removing the dust and/or odors (pollutants) in the room is based on extension of the conventional idea that a removal speed is enhanced by increasing an air flow volume of the air cleaner, there arises problems that a power consumption is increased and a noise emitted during an operation of the air cleaner is also increased.

In addition, there has been a desire to cause fine particles of active ingredients released from the air cleaner, which are, for example, water vapor, ions, and aromatic components, to reach every nook and corner of a room and to diffuse the active ingredients in the room. Since a method for realizing this desire is also based on the extension of the conventional idea that a speed of diffusing the active ingredients is enhanced by increasing an air flow volume of the air cleaner, as mentioned above, there arises the problems that the power consumption is increased and the noise emitted during the operation of the air cleaner is also increased.

Furthermore, in the air cleaner disclosed in Japanese Patent Application Laid-Open Publication No. 2006-17343 (Patent Document 1), since an air current is sent out in the lateral direction and the forward direction, an influence of obstacles in a room, such as furniture, is easily exerted and it is difficult to sufficiently favorably circulate the air. In addition, since the air current is sent out in the lateral direction and the forward direction, dust on a floor surface is likely to be blown up. Further, due to uncomfortable draft feeling, wind stress is likely to be imposed on persons.

Moreover, in the air cleaner disclosed in Japanese Patent Application Laid-Open Publication No. 2006-57919 (Patent Document 2), an automatically movable louver placed in the blowout port and a sensor for detecting air pollution are required. In this air cleaner, it is difficult to make an optimum air current merely by causing the louver to automatically swing. Even if the optimum air current in the room is made by stopping the movable louver at a position where a predetermined or more amount of the air pollution is detected, it is difficult to cause timing at which air in a position where the air is stagnant reaches the sensor and timing at which the movable louver turns toward the above-mentioned position to coincide with each other, thereby making it difficult to efficiently collect the dust. In addition, if a wind direction is changed with the louver, a wind flow width is decreased, thereby incurring a problem that the air current is impaired.

In view of the above-mentioned situations, the inventors have thoroughly pursued an improvement of an indoor air environment and have pondered again. Then, it has turned out that there is room for improvement in the following which has continued to be overlooked all along in the study of the improvement in a range of the conventional idea.

In other words, in a case of a floor-standing type (and/or a desktop type) air cleaner, conventionally, cleaned air is sent out vertically upward from a blowout port of the air cleaner, thereby cleaning the air in a room. In addition, in order to prevent a wall surface from being soiled due to triboelectrification, the air cleaner is placed at some distance from the wall surface. Therefore, when the cleaned air is sent out vertically upward, the air collides against a ceiling, and a part thereof flows toward a rear surface of the air cleaner, circulates in a narrow space between the air cleaner and the wall surface on a side of the rear surface of the air cleaner, and is immediately sucked into the air cleaner. Since it is difficult for this air current flowing toward the rear surface of the air cleaner to spread in the whole room, it can be said that this air is uselessly sent out. In addition, an air flow volume spreading in the whole room is decreased by an air flow volume of the air current flowing toward the rear surface, thereby leading to problems that a dust collecting capability is impaired and a distance over which an air current can reach is shortened. In other words, the conventional technologies have a problem that a large loss in circulation of the air current is caused, that is, an efficiency of the circulation of the air current is inferior and less effect of improving the indoor air environment is attained. In order to enhance the effect of improving the indoor air environment, the air flow volume may be increased by increasing the number of revolutions of a fan. However, if the air flow volume is increased by increasing the number of the revolutions of the fan, a new problem that a noise is increased arises.

An object of the present invention is to provide an air cleaner which is capable of enhancing an effect of improving an indoor air environment by enhancing an efficiency of circulating an air current without increasing an air flow volume.

### Means for Solving the Problems

An air cleaner according to the present invention is placed on a floor or a desk and used so as to be located in a vicinity of a side wall surface of a room, comprising: a main body; a suction port provided in the main body and for taking in air in the room; a removing part provided in the main body and for removing dust and/or a substance present in the air taken in from the suction port; a blowout port provided in the main body and for sending out the air treated by the removing part into the room; and an air blowing part provided in the main body and for moving the air from the suction port to the blowout port. When a distance from a middle position of the blowout port to the side wall surface of the room is supposed to be L [mm], a distance from the middle position of the blowout port to a ceiling surface of the room is supposed to be H [mm], and the air cleaner is placed and used in a position which allows the distance L to be a value selected from among values in a range of 100<L<600, in order to cause the air sent out from the blowout port to first reach the side wall surface of the room, an angle θ[°] formed between a direction in which the air is sent out from the blowout port and a vertically upward direction is set to be in a range of tan⁻¹(L/H)<θ≦35.

By employing the above-mentioned configuration, in the air cleaner placed on the floor or the desk and located in the vicinity of the side wall surface of the room, the angle of the air current of the cleaned air blown out is set so as to allow the cleaned air to be sent out from the blowout port at a predetermined angle with respect to the vertically upward direction, to be caused to reach the side wall surface of the room, to be next caused to flow along the side wall surface of the room, and to be further circulated along a ceiling and the other side wall surfaces and so as to allow the air in the room to be sucked from the suction port. In this configuration, an amount of the air current sent out from the blowout port and immediately sucked into the air cleaner is decreased and spreading, of the air current sent out from the blowout port, in a lateral direction is suppressed, thereby drastically extending a distance over which the air current can reach and drastically increasing an air velocity in a position in the room, which is away from the air cleaner. In addition, an air velocity in the vicinity of the wall surface of the room is also drastically increased.

As a result, an effect of improving an air environment in the position in the room, away from the air cleaner, can be enhanced and an indoor air in a wide range can be effectively cleaned. In addition, since an air current uselessly sent out can be decreased, even when an amount of an air flow volume is small, a desired effect of improving the air environment can be attained. Since the same effect of improving the air environment can be realized even with a small amount of the air flow volume, a noise can be reduced. In addition, in a case where gaps are present in windows and others provided in wall surfaces and airtightness of the room is low, since the air velocity in the vicinity of the wall surface of the room can be increased, a positive pressure effect can be attained and an amount of dust intruding into the room from an outside of the room can be reduced.

It is preferable that the air cleaner according to the present invention further comprises a blowout flow passageway for flowing the air from the air blowing part to the blowout port and the blowout flow passageway is arranged so as to be inclined toward the side wall surface of the room and used.

By employing the above-mentioned configuration, an angle at which an air current is bent at a position of the blowout port of the air cleaner toward the side wall surface side of the room can be decreased, thereby allowing a pressure loss in the blowout flow passageway to be reduced, improving a power consumption, and allowing a noise to be reduced. Further, sound emitted from the blowout port does not propagate directly into a housing region of a user, thereby allowing an indoor air to be effectively cleaned and a noise to be reduced.

It is preferable that the air cleaner according to the present invention further comprises a blowout flow passageway for flowing the air from the air blowing part to the blowout port and the blowout flow passageway is configured so as to be inclined at an angle OH fixed as one value selected from among values in a range of tan⁻¹(L/H)<θ≦35, the angle θ[°] formed between a direction of extension of a middle position of the blowout flow passageway and the vertically upward direction.

By employing the above-mentioned configuration, it is made unnecessary to bend the air current at a position of the blowout port of the air cleaner toward the side wall surface of the room, thereby allowing the pressure loss in the blowout flow passageway to be reduced, further improving the power consumption, and further allowing the noise to be reduced. Further, the sound emitted from the blowout port does not propagate directly into the housing region of a user, thereby allowing the indoor air to be effectively cleaned and the noise to be reduced.

It is preferable that in the air cleaner according to the present invention, the air blowing part includes a sirocco fan, and when a height of the sirocco fan is supposed to be t, a length, in a direction along the air current, of one wall surface among wall surfaces forming the blowout flow passageway is set to be t/sin θ or more, the one wall surface arranged in a position farthest from the side wall surface of the room.

By employing the above-mentioned configuration, since the sirocco fan is invisible from the blowout port, viewed from vertically above, before a noise generated from the air blower is emitted from the blowout port, the noise is reflected by the wall surface at least one time in the blowout flow passageway. When the sound is subjected to the reflection and interference, energy of the sound is attenuated, thereby allowing the sound emitted from the blowout port to be suppressed and the noise to be reduced.

It is preferable that in the air cleaner according to the present invention, a cross-sectional area of the blowout flow passageway gradually increases at a predetermined ratio in accordance with an increase in proximity to the blowout port. For example, in a case where a depth dimension of the blowout flow passageway is constant, it is preferable that a cross-sectional area of the blowout flow passageway gradually increases at an angle of approximately 20° in accordance with an increase in proximity to the blowout port, that is, an increase in proximity to a downstream part of the air current.

By employing the above-mentioned configuration, exfoliation of the air current along the wall surface of the blowout flow passageway is suppressed. Thus, a flow passageway resistance is small and kinetic energy of the air current is efficiently converted to a static pressure. In other words, a part of work done by the air blower (fan), which is a static pressure rise, is covered by the static pressure generated when the kinetic energy is converted as mentioned above, whereby energy required for the work of the air blower (fan) can be reduced and the air blower (fan) can be caused to do desired work with a low power consumption.

Further, it is preferable that in the air cleaner according to the present invention, a width along a direction of inclination of the blowout flow passageway with respect to the vertically upward direction gradually shrinks in accordance with an increase in proximity to the blowout port. In other words, a depth dimension of the blowout flow passageway gradually shrinks in accordance with an increase in proximity to the blowout port, that is, an increase in proximity to a downstream part thereof and the blowout flow passageway has a shape which gradually becomes narrowed in accordance with the increase in the proximity to the downstream part thereof.

By employing the above-mentioned configuration, since the blowout flow passageway has the shape which gradually becomes narrowed, an aspect ratio of the cross-sectional area of the blowout port is increased and an aspect ratio of a cross section of the air current sent out from the blowout port is accordingly increased. At this time, since even if a cross-sectional area through which the air current flows is the same, a ratio at which the air current contacts the wall surfaces is increased, an influence of surrounding air currents is hardly exerted. Therefore, since a flow converges, a potential core of the air current is extended. This extends a distance over which the air current can reach. In other words, the above-mentioned configuration allows the kinetic energy of the air current to be converted to the static pressure and the flow velocity of the air current to be reduced, thereby enabling extension of the distance over which such an air current can reach.

It is preferable that the air cleaner according to the present invention further comprises an operation part for operating the air cleaner, the suction port is arranged on a rear surface of the main body, the blowout port is arranged on a top surface of the main body, and the operation part is arranged on a front surface of the main body.

By employing the above-mentioned configuration, the operation part of the air cleaner is arranged on the front surface side of the main body and the suction port is arranged on the rear surface of the main body, thereby allowing a user to unwittingly place the air cleaner such that the suction port of the air cleaner faces a side of the side wall surface of the room.

It is preferable that the air cleaner according to the present invention is placed so as to face the side wall surface of the room and used.

By employing the above-mentioned configuration, sound emitted from the suction port is caused not to propagate directly into a housing region of a user, thereby allowing an indoor air to be effectively cleaned and a noise to be reduced.

It is preferable that the air cleaner according to the present invention is used in a room satisfying Q/A<0.35 when an air flow volume of the air sent out from the blowout port is supposed to be Q [m³/min] and an area of the floor is supposed to be A [m²].

By employing the above-mentioned configuration, the air current sent out from the blowout port travels along the wall surface of the room and the distance over which the air current can reach is extended, thereby allowing the above-described effect to be attained in an ensured manner.

### EFFECT OF THE INVENTION

As described above, according to the present invention, an effect of improving an air environment in a position in a room, away from an air cleaner, can be enhanced and an indoor air in a wide range can be effectively cleaned. In addition, since an air current uselessly sent out can be decreased, even when an amount of an air flow volume is small, a desired effect of improving the air environment can be attained. Since the same effect of improving the air environment can be realized even with a small amount of the air flow volume, a noise can be reduced. Further, in a case where gaps are present in windows and others provided in wall surfaces and airtightness of the room is low, an air velocity in the vicinity of a wall surface of the room can be increased, thereby attaining a positive pressure effect and allowing a reduction in an amount of dust intruding into the room from an outside of the room.

### BRIEF DESCRIPTION OF THE DRAWINGS

FIG. 1 is a side sectional view schematically illustrating an air cleaner of an embodiment 1-1.

FIG. 2 is a partial side sectional view illustrating a blowout flow passageway of the air cleaner shown in FIG. 1 in an expanded manner.

FIG. 3 is a cross-sectional view (A) illustrating the blowout flow passageway, taken from a IIIA direction in FIG. 2 and a cross-sectional view (B) showing a relationship of angles in the blowout flow passageway shown in FIG. 2.

FIG. 4 is a diagram illustrating placement of the air cleaner and a measurement position where dust collecting performance in a room, attained by the air cleaner, is measured.

FIG. 5 is a diagram schematically illustrating a highly airtight room (A) and a less airtight room (B) used for measuring the dust collecting performance.

FIG. 6 is a graph showing, as a result of the measurement of the dust collecting performance, attenuation of dust concentrations in a measurement position, shown in FIG. 4, which is far away in a direction in which a front surface of each of the air cleaners faces in a case where the air cleaner according to the present invention and a conventional air cleaner were operated in the highly airtight room, shown in (A) of FIG. 5, which had a size corresponding to approximately 30 tatami mats.

FIG. 7 is a graph showing attenuation of dust concentrations in the measurement position, shown in FIG. 4, which is far away in the direction in which the front surface of each of the air cleaners faces in a case where the air cleaner according to the present invention and the conventional air cleaner were operated in the less airtight room, shown in (B) of FIG. 5, which had the size corresponding to approximately 30 tatami-mats.

FIG. 8 is a diagram illustrating placement of the air cleaner and measurement positions where air velocities attained by the air cleaner are measured.

FIG. 9 is a graph showing a result of measuring the air velocity.

FIG. 10 is a diagram illustrating placement of the air cleaner and measurement positions where air velocities attained by the air cleaner are measured.

FIG. 11 is a graph showing a result of measuring the air velocities in a traveling direction measurement position and a lateral direction measurement position.

FIG. 12 is a diagram illustrating placement of the air cleaner and a measurement position where an air velocity attained by the air cleaner is measured.

FIG. 13 is a graph showing a result of measuring the air velocity.

FIG. 14 is a cross-sectional view schematically illustrating an indoor air current in a room in which a conventional air cleaner is used.

FIG. 15 is a development view schematically illustrating the indoor air current (air current along the wall surface) sent out from the conventional air cleaner.

FIG. 16 is a cross-sectional view schematically illustrating an indoor air current in a room in which the air cleaner according to the present invention is used.

FIG. 17 is a development view schematically illustrating the indoor air current (air current along the wall surface) sent out from the air cleaner according to the present invention.

FIG. 18 is a diagram showing a result obtained when in each case of the air current sent out from the conventional air cleaner and the air current sent out from the air cleaner according to the present invention, a path of the air current circulating in a space of a room is visualized by using a CFD (Computational Fluid Dynamics) analysis (computer simulation).

FIG. 19 is a cross-sectional view schematically illustrating the vicinities of gaps in a case where the conventional air cleaner is placed in the less airtight room.

FIG. 20 is a cross-sectional view schematically illustrating the vicinities of gaps in a case where the air cleaner according to the present invention is placed in the less airtight room.

FIG. 21 is a graph showing a temporal change in a dust concentration, caused when the conventional air cleaner was operated.

FIG. 22 is a graph showing a temporal change in a dust concentration, caused when the air cleaner according to the present invention was operated.

FIG. 23 is a cross-sectional view schematically illustrating a relationship between a height of a sirocco fan and a length of a front wall forming a blowout flow passageway.

FIG. 24 is a side cross-sectional view schematically illustrating an air cleaner of an embodiment 1-2.

FIG. 25 is a side cross-sectional view schematically illustrating an air cleaner of an embodiment 2-1.

FIG. 26 is a side cross-sectional view schematically illustrating an air cleaner of an embodiment 2-2.

FIG. 27 is a side cross-sectional view schematically illustrating an air cleaner of an embodiment 2-3.

FIG. 28 is a side cross-sectional view schematically illustrating an air cleaner of an embodiment 3-1.

FIG. 29 is a side cross-sectional view schematically illustrating an air cleaner of an embodiment 3-2.

FIG. 30 is a side cross-sectional view schematically illustrating an air cleaner of the embodiment 4-1.

FIG. 31 is a side cross-sectional view schematically illustrating an air cleaner of an embodiment 4-2.

FIG. 32 is a side cross-sectional view schematically illustrating an air cleaner of a comparative embodiment according to the present invention.

FIG. 33 is a graph showing a relationship between an air flow volume and a noise in each of the air cleaners.

FIG. 34 is a side sectional view schematically illustrating one example of the conventional air cleaner.

FIG. 35 is a side sectional view schematically illustrating another example of the conventional air cleaner.

### EXPLANATION OF REFERENCE NUMERALS

1: air cleaner, 100: housing, 110: front surface, 120: rear surface, 130: top surface, 140: bottom surface, 122: suction port, 131: blowout port, 150: air filter, 170: air blower, 171: sirocco fan 180: blowout flow passageway, 190: operation part, W1: side wall surface.

### BEST MODE FOR CARRYING OUT THE INVENTION

### First Embodiment

### Embodiment 1-1

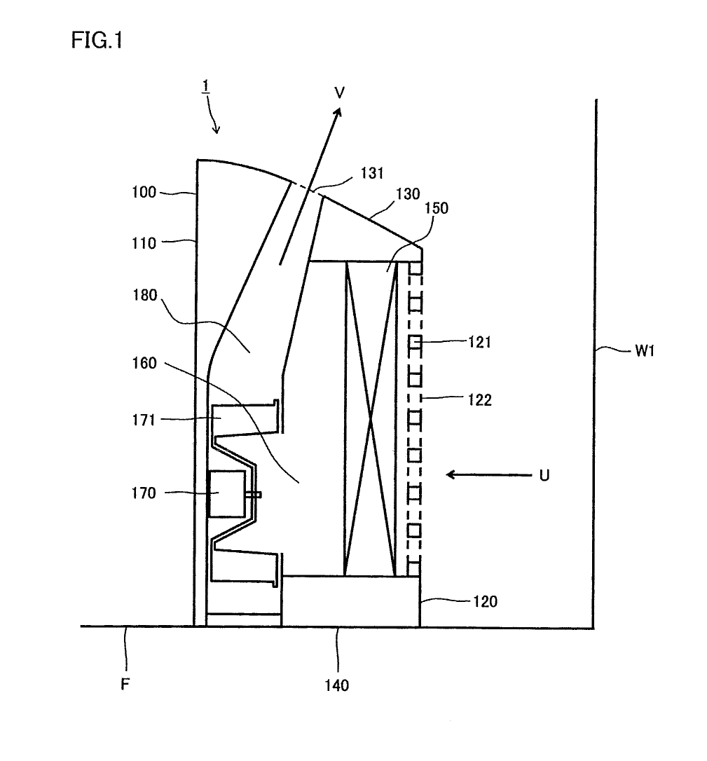

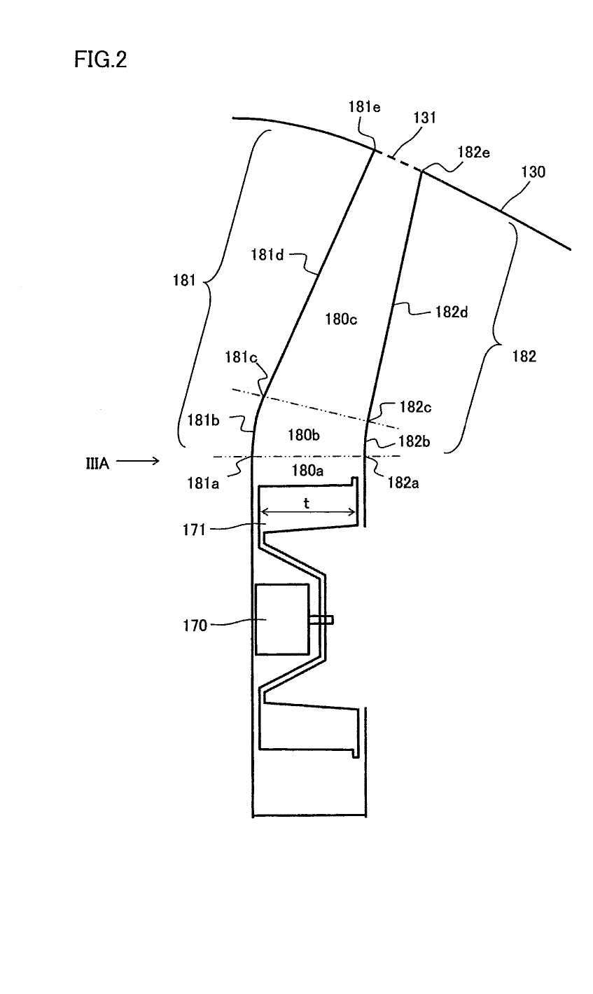

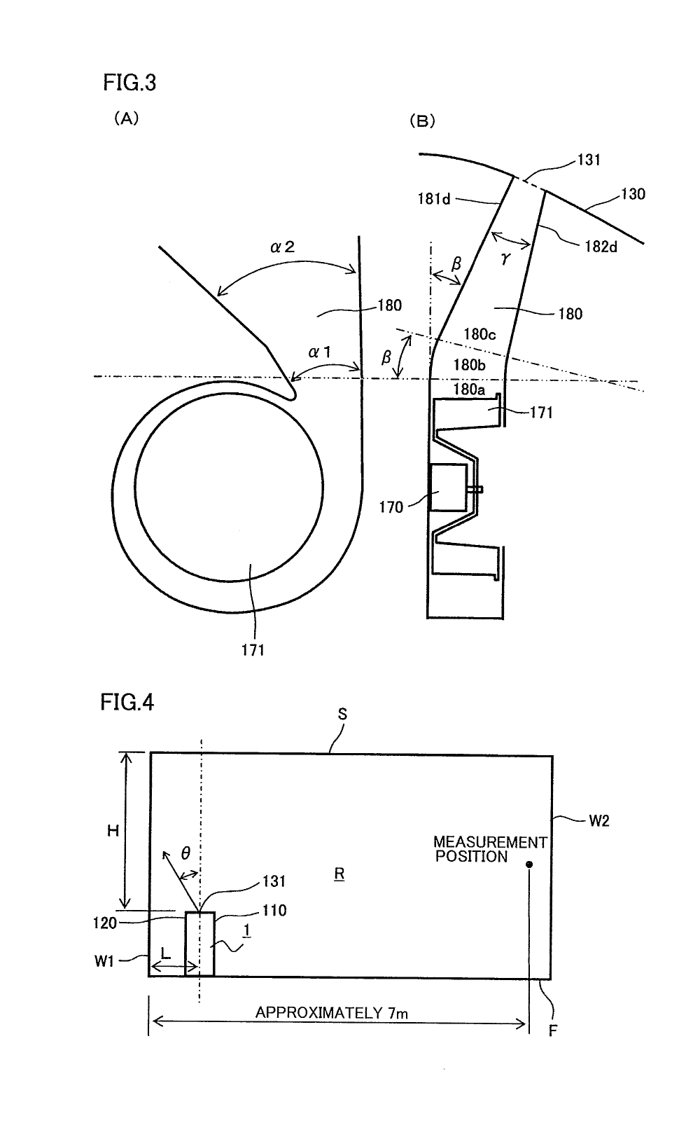

Hereinafter, an embodiment 1-1 of the present invention will be described with reference to drawings. FIG. 1 is a side sectional view schematically illustrating an air cleaner of the present embodiment. FIG. 2 is a partial side sectional view illustrating a blowout flow passageway of the air cleaner shown in FIG. 1 in an expanded manner. FIG. 3 is a cross-sectional view (A) illustrating the blowout flow passageway, taken from a IIIA direction in FIG. 2 and a cross-sectional view (B) showing a relationship of angles in the blowout flow passageway shown in FIG. 2.

As shown in FIG. 1, the air cleaner 1 is placed on a floor surface F or a desk and used so as to be located in the vicinity of a side wall surface W1 of a room. The air cleaner 1 comprises: a main body; a suction port 122 provided in the main body and for taking in air inside the room; an air filter 150 provided in the main body as a removing part for removing dust and/or a substance present in the air taken in from the suction port 122; a blowout port 131 provided in the main body and for sending out the air treated by the air filter 150 into the room; and an air blower 170 provided in the main body as an air blowing part for moving the air from the suction port 122 to the blowout port 131.

The main body of the air cleaner 1 is held by a housing 100. The housing 100 is placed on the floor surface F and has a front surface (anterior surface) 110, a rear surface (posterior surface) 120, a top surface (upper surface) 130, and a bottom surface 140 contacting the floor surface F. The rear surface 120 of the housing 100 is covered by a perforated panel 121 in which a multitude of suction ports 122 are provided in a grid-like manner. Here, it is supposed that a side of the air cleaner 1, from which cleaned air is sent out in a direction indicated by an arrow V is a side of the rear surface 120 and a side opposite thereto is a side of the front surface 110, with the air cleaner 1 viewed from vertically above.

The housing 100 is placed on the floor surface F so as to cause the side of the rear surface 120 to face the side wall surface W1 of the room and to be located at a distance of, for example, 300 mm from the side wall surface W1. On the top surface 130 of the housing 100, the blowout port 131 is provided. The blowout port 131 is formed so as to be of a substantially rectangular shape extending in a width direction of the housing 100 and is provided so as to face backwardly upward.

Inside the housing 100, an air flow channel which communicates from the suction port 122 to the blowout port 131 and includes a suction flow passageway 160 and a blowout flow passageway 180 is formed. Inside the air flow channel, the air blower 170 for sending out the air is placed. As a fan constituting the air blower 170, a sirocco fan 171 is used. In the air flow channel, on a downstream side of the sirocco fan 171, the blowout flow passageway 180 for flowing the air from the sirocco fan 171 to the blowout port 131 is formed. The blowout flow passageway 180 guides the air sent out by the sirocco fan 171 backwardly upward. A cross-sectional area of the blowout flow passageway 180 gradually increases in accordance with an increase in proximity to the blowout port 131 and an increase in proximity to a downstream part thereof. As shown in FIG. 2, the blowout flow passageway 180 communicates from the air blower 170 to the blowout port 131 and is formed so as to include an upward guide part 180a, an inclination part 180b, and a backwardly upward guide part 180c.

As shown in FIG. 2, a front wall 181 is formed so as to have as a starting point a terminal 181a of the upward guide part 180a, to extend along a front surface 181b of the inclination part 180b, to connect to the backwardly upward guide part 180c at a terminal position 181c of the inclination part 180b, to extend along a backwardly upward inclination part 181d which is formed such that an backward inclination thereof increases from a connecting portion 181c thereof to the blowout port 131 in accordance with an increase in proximity to an upper part thereof, and to have as an end point an intersection point 181e with the top surface 130 of the housing 100. When a height of the sirocco fan 171 is supposed to be t, a length of the front wall 181 (a length from the starting point 181a to the end point 181e) in a direction along a slope thereof is set to be, for example, 4.1t. In addition, the end point 181e of the front wall 181 is set so as to be located behind the end point 182a of the upward guide part 180a.

A rear wall 182 is formed so as to have as a starting point a terminal 182a of the upward guide part 180a, to extend along a rear surface 182b of the inclination part 180b, to connect to the backwardly upward guide part 180c at a terminal position 182c of the inclination part 180b, to form a backwardly upward inclination part 182d which is formed such that an backward inclination thereof gradually increases from a connecting portion 182c thereof to the blowout port 131 in accordance with an increase in proximity to an upper part thereof, such that a spacing with the front wall 181 decreases, and such that a cross section of a flow passageway thereof gradually becomes narrowed in accordance with an increase in proximity to the upper part thereof, and to have as an end point an intersection point 182e with the top surface 130 of the housing 100. When the height of the sirocco fan 171 is supposed to be t, a length of the rear wall 182 (a length from the starting point 182a to the end point 182e) in a direction along a slope thereof is set to be, for example, 3.8t.

The blowout flow passageway 180 constituting the air flow channel is set as shown in FIG. 3. Specifically, depth dimensions of the upward guide part 180a and the inclination part 180b are set to be constant; a flow passageway open angle α1 is set to be 20°; a flow passageway open angle α2 of the backwardly upward guide part 180c is set to be 40°; an angle (angle formed by the front wall and a plane perpendicular to a side cross-section and the ground) B, formed by a plane where the upward guide part 180a abuts the inclination part 180b and a plane where the inclination part 180b abuts the backwardly upward guide part 180c, is set to be 20°; and an angle γ formed by the backwardly upward inclination part 181d on a front surface side of the backwardly upward guide part 180c and the backwardly upward inclination part 182d on a rear surface side thereof is set to be 8°. In addition, a joining portion of the upward guide part 180a and the inclination part 180b as well as a joining portion of the inclination part 180b and the backwardly upward guide part 180c are joined so as to abut each other, respectively or so as to be smoothly connected to each other, respectively. It is preferable that the above-mentioned α1 is set to be approximately 10° through 30°, the above-mentioned β is set to be approximately 15° through 35°, and the above-mentioned γ is set to be approximately 0° through 15°. By making the settings as described above, exfoliation of an air current in each part is suppressed to a minimum and the air current smoothly flows without drastically increasing a flow passageway resistance.

In addition, in a case where the housing 100 is placed on the floor surface F such that a side of the rear surface 120 thereof faces the side wall surface W1 of the room and a spacing of 100 mm from the side wall surface W1 is provided, it is preferable that 6 is set to be approximately 10° through 25°.

In a position facing the perforated panel 121, the air filter 150 for collecting and removing dust and/or odors (pollutants) present in the air sucked from the suction port 122 is provided. In the suction flow passageway 160 between the sirocco fan 171 and the air filter 150 in the air flow channel, a humidifier (not shown) may be provided. In a case where the humidifier (not shown) is provided, due to a large pressure loss (air flow resistance) particularly in a vaporization-type humidifier, work required for raising a static pressure by using the air blower is drastically increased, whereby in order to obtain a desired air flow volume, the number of rotations is required to be drastically increased and a noise caused during an operation of the air cleaner is also drastically increased. In the air cleaner according to the present embodiment, since a reduction in the noise can be realized, the air cleaner can be provided with the humidifier without increasing the noise caused during the operation. In the blowout flow passageway 180 between the sirocco fan 171 and the blowout port 131 in the air flow channel, an ion generator (not shown) may be provided.

Upon starting an operation of the air cleaner 1 configured as described above, as shown in FIG. 1, the sirocco fan 171 of the air blower 170 is driven in a rotated manner, air is sucked from the suction port 122, and the dust and/or odors (pollutants) present in the air is removed by the air filter 150.

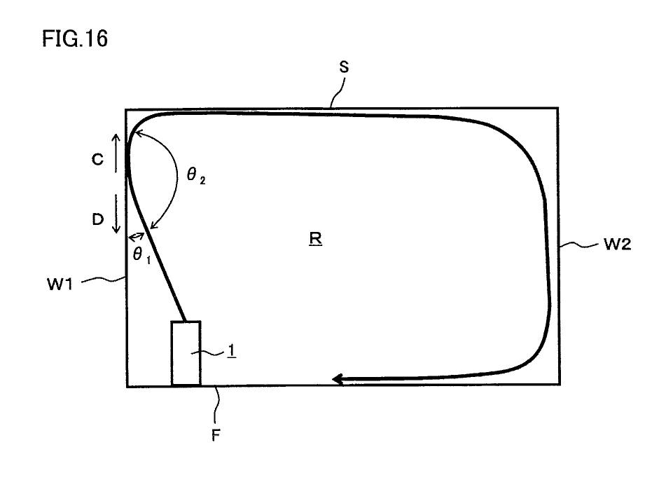

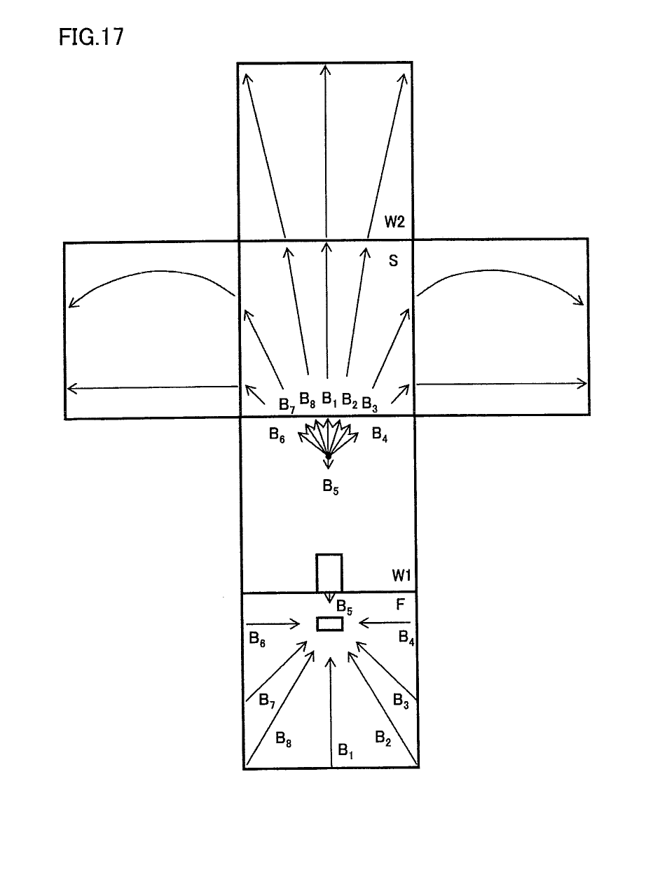

A direction of a flow of the cleaned air which has been taken in the housing 100 and has had therefrom the dust removed by the air filter 150 is regulated by the sirocco fan 171 and the blowout flow passageway 180, and the cleaned air smoothly flows along the front wall 181, whose backward inclination gradually increases in accordance with the increase in the proximity to the upper part from the inclination part 180b of the blowout flow passageway 180, and is sent out into the room backwardly upward in the direction indicated by the arrow V shown in FIG. 1. This causes the air cleaner 1 to send out the cleaned air backwardly upward. As shown in FIG. 16, the cleaned air sent out into the room reaches the side wall surface W1 of the room R. Thereafter, due to the Coanda effect, the cleaned air flows sequentially from the side wall surface W1 along a ceiling surface S, a side wall surface W2 facing the air cleaner 1, and the floor surface F and is sucked from both lateral sides of the air cleaner 1 to the suction ports 122 placed on the side of the rear surface 120.

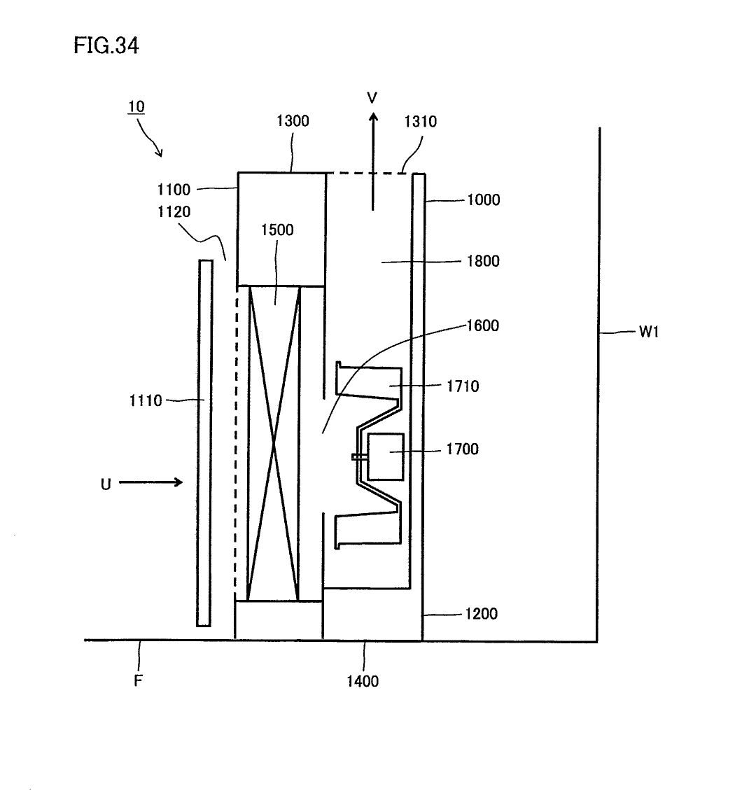

By using the air cleaner 1 according to the present invention shown in FIG. 1 through FIG. 3 and the conventional air cleaner shown in FIG. 34, dust collecting performance was measured. The dust collecting performance was measured in a highly airtight and a less airtight room.

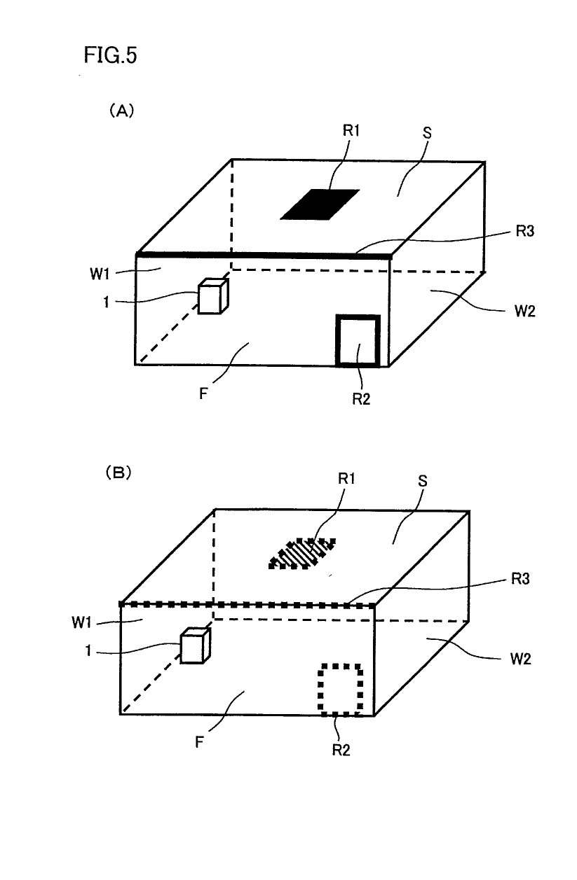

FIG. 4 is a diagram illustrating placement of the air cleaner and a measurement position where the dust collecting performance in a room, attained by the air cleaner, is measured.

As shown in FIG. 4, the air cleaner 1 according to the present invention (or the conventional air cleaner 10) was placed in the room R as described below. A distance L from a middle position of the blowout port 131 (or the blowout port 1310) to the side wall surface W1 of the room was 370 mm and a distance H from the middle position of the blowout port 131 (or the blowout port 1310) to the ceiling surface S of the room was 2380 mm. In the air cleaner 1 according to the present invention, an angle θ formed between a direction of a flow of the air sent out from the blowout port 131 and a vertically upward direction was 20° which was an angle fixed as one value selected from among values in a range of tan⁻¹(L/H)<θ≦35 (or an angle θ formed between a direction in which a middle position of the blowout flow passageway 180 extends and the vertically upward direction was 20° which was an angle fixed as one value selected among from values in a range of tan⁻¹(L/H)<θ≦35). In the conventional air cleaner 10, an angle θ formed between a direction of a flow of the air sent out from the blowout port 1310 and a vertically upward direction was 0°, that is, the direction of the flow of the air sent out from the blowout port 1310 was the vertically upward direction. The measurement position where the dust collecting performance in the room was measured was in a central portion of the side wall surface W2 facing the side wall surface W1 and located at a distance of approximately 7 m from the side wall surface W1. A volume of the air sent out from the air cleaner was 6.2 m³/min.

FIG. 5 is a diagram schematically illustrating the highly airtight room (A) and the less airtight room (B) used for measuring the dust collecting performance.

As shown in (A) of FIG. 5, in the highly airtight room, a gap R1 in the ceiling surface S on which an air conditioner was installed, a gap R2 in a door, and a gap R3 between the ceiling surface S and the side wall surface W1 were sealed up, thereby virtually eliminating the gaps through which an inside and an outside of the room communicate with each other. On the other hand, as shown in (B) of FIG. 5, in the less airtight room, the above-mentioned gaps R1, R2 and R3 were not sealed up. As measurement conditions, in the highly airtight room, a dust concentration inside the room was higher than a dust concentration outside the room; and in the less airtight room, a dust concentration inside the room was lower than a dust concentration outside the room.

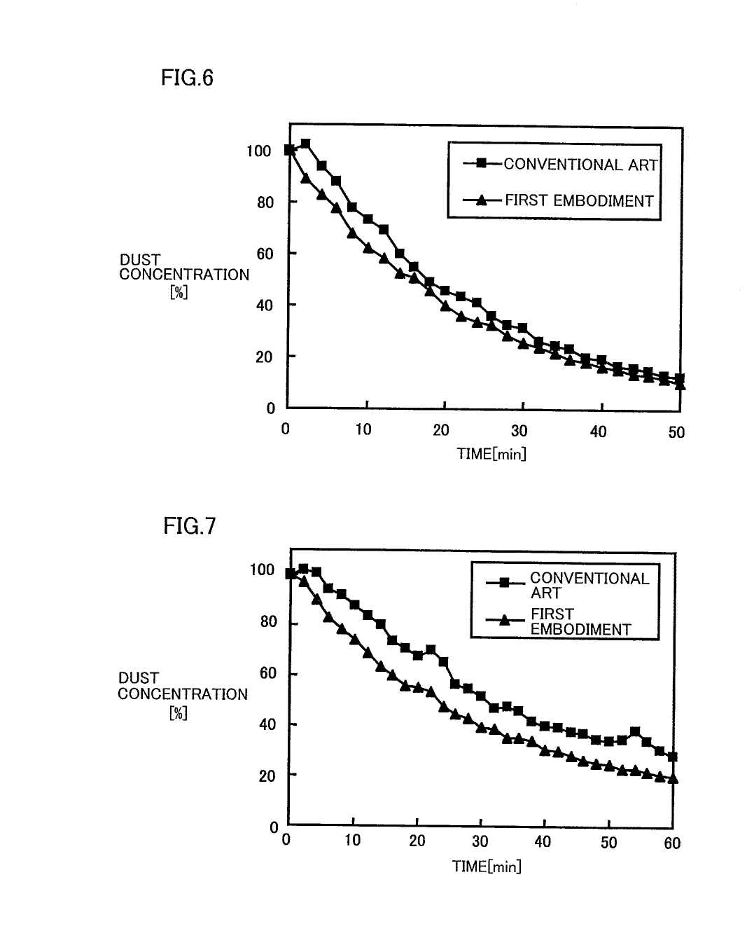

FIG. 6 is a graph showing, as a result of the measurement of the dust collecting performance, attenuation of the dust concentrations in the measurement position, shown in FIG. 4, which was far away in a direction in which the front surface of each of the air cleaners faced in a case where the air cleaner 1 according to the present invention (▴ in FIG. 6) and the conventional air cleaner 10 (▪ in FIG. 6) were operated in the highly airtight room, shown in (A) of FIG. 5, which had a size corresponding to approximately 30 tatami mats. A horizontal axis indicates time (unit: min) which passed from when each of the air cleaners was turned on and a vertical axis indicates each of the dust concentrations (unit: %) in the room. It is seen from FIG. 6 that the air cleaner 1 according to the present invention is capable of more quickly and effectively cleaning the air in the room, particularly in the position far away from the air cleaner, than the conventional air cleaner 10. In addition, it is seen therefrom that time required when the air cleaner 1 according to the present invention attained a particular dust concentration is approximately 3 minutes shorter than time required when the conventional air cleaner 10 attained the same dust concentration as that particular dust concentration attained by the air cleaner 1 according to the present invention.

In addition, FIG. 7 is a graph showing, as a result of the measurement of the dust collecting performance, attenuation of the dust concentrations in the measurement position, shown in FIG. 4, which was far away in a direction in which the front surface of each of the air cleaners faced in a case where the air cleaner 1 according to the present invention (▴ in FIG. 7) and the conventional air cleaner 10 (▪ in FIG. 7) were operated in the less airtight room, shown in (B) of FIG. 5, which had a size corresponding to approximately 30 tatami mats. A vertical axis and a horizontal axis indicate the same as in FIG. 6. It is seen from FIG. 7 that the air cleaner 1 according to the present invention is capable of more quickly and effectively cleaning the air in the room, particularly in the position far away from the air cleaner, than the conventional air cleaner 10. In addition, it is seen therefrom that time required when the air cleaner 1 according to the present invention attained a particular dust concentration is more shortened in accordance with an increase in a lapse of time than time required when the conventional air cleaner 10 attained the same dust concentration as that particular dust concentration attained by the air cleaner 1 according to the present invention. In other words, in the less airtight room rather than in the highly airtight room, the air cleaner 1 according to the present invention exhibits great superiority in an air cleaning effect over the conventional air cleaner 10.

As described above, according to the present invention, the air in the room R and, in particular, the air which is away from the air cleaner 1 and in the vicinity of the side wall surface W2 can be effectively cleaned.

Next, a mechanism with which the dust collecting performance of the air cleaner according to the present invention is enhanced as shown in the above-mentioned FIG. 6 and FIG. 7 will be described.

First, what change the configuration of the present invention brings about in an air current circulating in a room will be described based on the below-described experimental result.

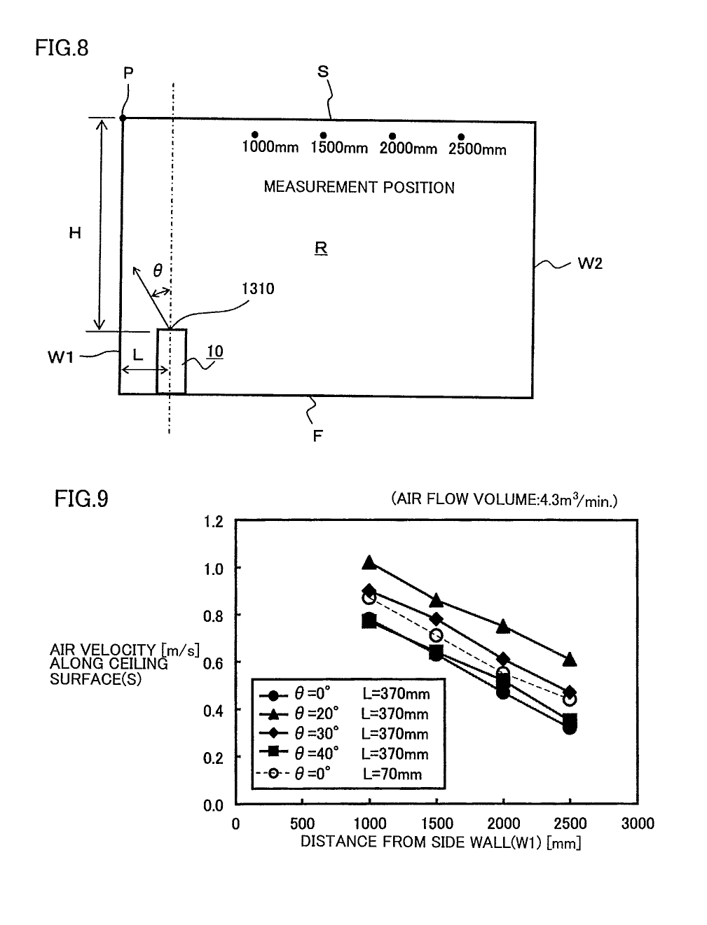

FIG. 8 is a diagram illustrating placement of the air cleaner and measurement positions where an air velocity attained by the air cleaner is measured.

As shown in FIG. 8, the conventional air cleaner 10 shown in FIG. 34 was placed in the room R as described below. A distance L from the middle position of the blowout port 1310 to the side wall surface W1 was 370 mm or 70 mm and a distance H from the middle position of the blowout port 1310 to the ceiling surface S of the room was 1880 mm. In the conventional air cleaner 10, an angle θ formed between a direction of a flow of the air sent out from the blowout port 1310 and the vertically upward direction was 0°, 20°, 30°, or 40°. When the conventional air cleaner 10 placed as described above was operated, the air velocity along the ceiling surface S was measured. The measurement positions where the air velocity was measured were four positions on the ceiling surface S, which were 1000 mm, 1500 mm, 2000 mm, and 2500 mm away from the side wall surface W1, respectively. An air flow volume of the air sent out from the air cleaner was 4.3 m³/min.

FIG. 9 is a graph showing a result of measuring the air velocity. A horizontal axis indicates distances (unit: mm) from the side wall, as the measurement positions and a vertical axis indicates the air velocity (unit: m/s) along the ceiling surface, in each of the measurement positions.

Based on the measurement result shown in FIG. 9, first, when the air cleaner 10 was placed on the floor surface F so as to provide a spacing of a distance 300 mm from the side wall surface W1 of the room (L=370 mm), a case where an air current was sent out from the blowout port 1310 of the air cleaner 10 vertically upward (θ=0°) ( in FIG. 9) was compared with a case where the air current was sent out from the blowout port 1310 of the air cleaner 10 obliquely backward (θ=20°) (▴ in FIG. 9), a case where the air current was sent out from the blowout port 1310 of the air cleaner 10 obliquely backward (θ=30°) (♦ in FIG. 9), and a case where the air current was sent out from the blowout port 1310 of the air cleaner 10 obliquely backward (θ=40°) (▪ in FIG. 9). It is seen from FIG. 9 that by making the setting in which the air current was sent out from the blowout port 1310 obliquely backward so as to allow θ to be approximately 20° through 30°, an air velocity in the vicinity of the ceiling surface S was drastically increased, as compared with the case where the air current was sent out therefrom vertically upward (θ=0°). In addition, when θ was set to be approximately 40°, an air velocity in the vicinity of the ceiling surface S was substantially equivalent to that in the case where the air current was sent out therefrom vertically upward (θ=0°).

This phenomenon is made clear through the following comparison. First, when the air cleaner 10 was placed on the floor surface F so as to allow a side of the rear surface 1200 to face the side wall surface W1 of the room and the air current was sent out from the blowout port 1310 of the air cleaner 10 vertically upward (θ=0°), in FIG. 9, a case where the air cleaner 10 was placed so as to provide a spacing of a distance 300 mm from the side wall surface W1 of the room (in FIG. 9) was compared with a case where the air cleaner 10 was placed so as to provide a spacing of a distance 0 mm from the side wall surface W1 of the room and to allow the rear surface 1200 of the air cleaner 10 to abut the side wall surface W1 (L=70 mm) (∘ in FIG. 9). It is seen from FIG. 9 that as compared with the case where the air cleaner 10 was placed so as to provide the spacing of the distance 300 mm from the side wall surface W1 of the room ( in FIG. 9), an air velocity along the ceiling surface S on a side of the front surface 1100 of the air cleaner 10 in the case where the air cleaner 10 was placed so as to provide the spacing of the distance 0 mm from the side wall surface W1 of the room and to allow the rear surface 1200 of the air cleaner 10 to abut the side wall surface W1 was increased. This indicates that in the case where the air cleaner 10 was placed so as to provide the spacing of the distance 300 mm from the side wall surface W1, a part of the air current flowed toward the side of the rear surface 1200 of the air cleaner 10 and the air current flowing toward a side of the front surface 1100 thereof was impaired. In other words, the result shown in FIG. 9 bears out that depending on the position where the air cleaner is placed, a great influence is exerted on the air current in the room.

In addition, in FIG. 9, a case where the air cleaner 10 was placed so as to provide the spacing of the distance 0 mm from the side wall surface W1 of the room and to allow the rear surface 1200 of the air cleaner 10 to abut the side wall surface W1 and the air current was sent out from the blowout port 1310 of the air cleaner 10 vertically upward (θ=0°) (∘ in FIG. 9) was compared with a case where the air cleaner 10 was placed on the floor surface F so as to provide the spacing of the distance 300 mm from the side wall surface W1 of the room and the air current was sent out from the blowout port 1310 of the air cleaner 10 obliquely backward (θ=20°) (▴ in FIG. 9). It is seen from FIG. 9 that as compared with the case where the air cleaner 10 was placed so as to allow the rear surface 1200 of the air cleaner 10 to abut the side wall surface W1 and the air current was sent out from the blowout port 1310 of the air cleaner 10 vertically upward ((θ=0°)(∘ in FIG. 9), an air velocity, sent out from the air cleaner, along the ceiling surface S was increased in the case where the air cleaner 10 was placed so as to provide the spacing of the distance 300 mm from the side wall surface W1 and the air current was sent out from the blowout port 1310 of the air cleaner 10 obliquely backward (θ=20°) (▴ in FIG. 9). This is because in the case where the air cleaner 10 is placed so as to allow the rear surface 1200 of the air cleaner 10 to abut the side wall surface W1 and the air current is sent out vertically upward (θ=0°), although diffusion of the air current is suppressed by the side wall surface W1, the air current is affected by viscous drag of the side wall surface W1. In contrast to this, in the case where the air cleaner 10 is placed so as to provide the spacing of the distance 300 mm from the side wall surface W1 and the air current is sent out obliquely backward (θ=20°), since the diffusion of the air current is suppressed by the side wall surface W1 and the air current is not affected by the viscous drag of the side wall surface W1 until the air current collides with the side wall surface W1, the air velocity along the ceiling surface S on the side of the front surface 1100 of the air cleaner 10 is hardly impaired.

Furthermore, when the air cleaner 10 was placed on the floor surface F so as to provide the spacing of the distance 300 mm from the side wall surface W1, in FIG. 9, the case where the air current was sent out obliquely backward (θ=20°) (▴ in FIG. 9), the case where the air current was sent out obliquely backward (θ=30°) (♦ in FIG. 9), and the case where the air current was sent out obliquely backward (θ=40°) (▪ in FIG. 9) were compared. It is seen from FIG. 9 that the air velocity along the ceiling surface S was decreased in accordance with an increase in θ. This is because a distance reached by the air current along the side wall surface W1 is increased in accordance with the increase in θ and the air current is easily affected by the viscous drag of the side wall surface W1 and because kinetic energy of the air current is easily impaired when the air current collides with the side wall surface W1 and spreading of the air current in a lateral direction with respect to a traveling direction is increased (this is described later with reference to FIG. 11).

As described above, the result shown in FIG. 9 bears out that depending on the angle of the air current sent out from the blowout port and the position where the air cleaner is placed, a great influence is exerted on the air current in the room.

In the air cleaner according to the present invention, when the distance from the middle position of the blowout port to the side wall surface of the room is supposed to be L [mm], the distance from the middle position of the blowout port to the ceiling surface of the room is supposed to be H [mm], and the air cleaner is placed and used in a position which allows the distance L to be a value selected from among values in a range of 100<L<600, in order to cause the air sent out from the blowout port to first reach the side wall surface of the room, the angle θ[°] formed between the direction in which the air is sent out from the blowout port and the vertically upward direction is set to be in a range of tan⁻¹(L/H)<0<35.

Here, an upper limit of θis set to be 35° based on the result shown in FIG. 9. A lower limit of θ is set to be tan⁻¹(L/H) which is an angle, in FIG. 8, formed between the vertically upward direction and a straight line spanning between the middle position of the blowout port 1310 and a point P at which lines of the side wall surface W1 of the room R and ceiling surface S intersect. This is because if the angle θ is greater than or equal to tan⁻¹(L/H), the air sent out from the blowout port 1310 first collides with at least the side wall surface W1.

In a case where θ is less than 15°, since depending on a height of the ceiling surface S and a distance from the side wall surface W1, there may be a case where the dust collecting performance is drastically enhanced and a case where the dust collecting performance is drastically impaired, attention is required.

For example, in a case where the air cleaner is placed on the floor surface F so as to provide the spacing of 300 mm from the side wall surface W1 and the air current is sent out obliquely backward (θ=10°), the air current sent out from the blowout port first collides with the ceiling surface S, not the side wall surface W1. Since the air current collides with the ceiling surface S from an oblique direction, the air current hardly flows in a direction (toward the side of the front surface of the air cleaner) which forms an acute angle with respect to a plane with which the air current collides. In other words, since the air current easily flows in a direction (toward the side of the rear surface of the air cleaner) which forms an obtuse angle with respect to the plane with which the air current collides, the air current flowing toward the side of the front surface of the air cleaner is impaired, as compared with the case where the air current is sent out vertically upward (θ=0°). Specifically, a part of the air current flows toward a side of the rear surface of the air cleaner, circulates in narrow space between the side of the rear surface of the air cleaner and the side wall surface, and is immediately sucked into the air cleaner. Since it is difficult for this air current flowing toward the rear surface of the air cleaner to reach the whole room, it can be said that this air current is uselessly sent out. In addition, an air flow volume spreading in the whole room is decreased by an air flow volume of the air current flowing toward the side of the rear surface, thereby leading to problems that a dust collecting capability is impaired and a distance over which an air current can reach is shortened.

However, in a case where the air cleaner is placed on the floor surface F so as to provide a spacing of 100 mm from the side wall surface W1 and the air current is sent out obliquely backward (θ=10°), the air current sent out from the blowout port first collides with the side wall surface. Accordingly, in this case, an effect which is substantially equivalent to that attained in the case where the air cleaner is placed on the floor surface F so as to provide the spacing of 300 mm from the side wall surface W1 and the air current is sent out obliquely backward (θ=20°) can be obtained.

Next, an air current sent out from the blowout port of the conventional air cleaner was blown against the side wall surface W1 at an angle θ, and changes in respective air velocities in a position in the vicinity of the side wall surface W1 in a traveling direction of the air current and a position in the vicinity of the side wall surface W1 in a lateral direction orthogonal to the traveling direction thereof in the position where the air current is blown against the side wall surface W1, occurring when the above-mentioned angle θ was changed, were examined.

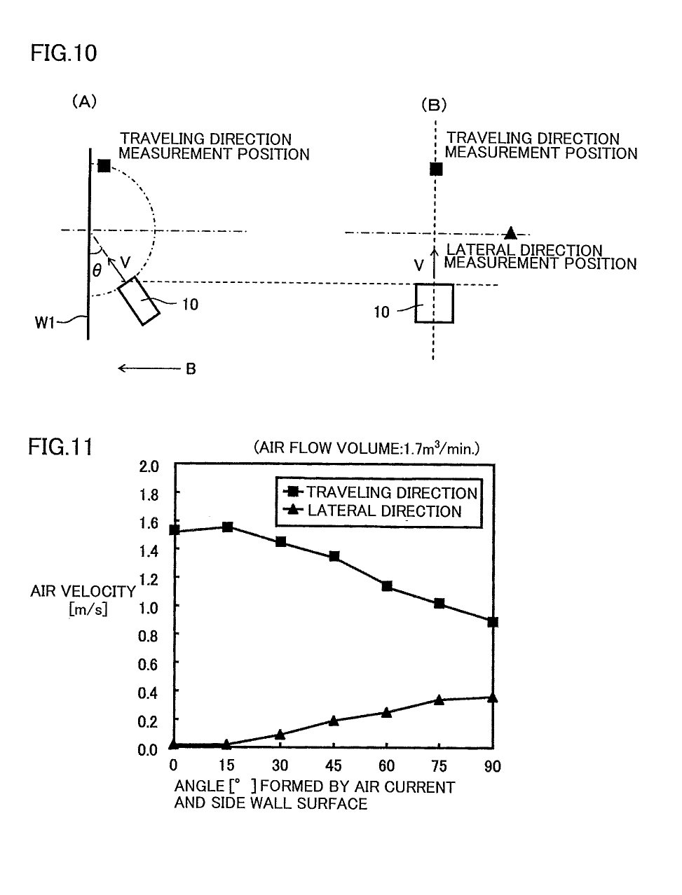

FIG. 10 is a diagram illustrating placement of the air cleaner and measurement positions where the air velocities attained by the air cleaner are measured. A diagram taken from a direction indicated by an arrow B in (A) of FIG. 10 is shown in (B).

As shown in FIG. 10, the conventional air cleaner 10 shown in FIG. 34 was placed in the vicinity of the side wall surface W1 and the angle θ at which the air current V sent out from the blowout port of the air cleaner 10 was blown against the side wall surface W1 was changed. As shown in (A) and (B) of FIG. 10, as the measurement positions of the air velocities, the position in the vicinity of the side wall surface W1 in the traveling direction of the air current was defined as a traveling direction measurement position ▪ and as shown in (B) of FIG. 10, the position in the vicinity of the side wall surface W1 in the lateral direction orthogonal to the traveling direction of the air current blown against the side wall surface W1 was defined as a lateral direction measurement position ▴.

FIG. 11 is a graph showing a result of measuring the air velocities in the traveling direction measurement position ▪ and the lateral direction measurement position ▴. A horizontal axis indicates an angle θ of the air current V blown against the side wall surface W1, that is, an angle θ (unit: °) formed by the air current V and the side wall surface W1, and a vertical axis indicates an air velocity (unit: m/s) attained at the angle θ. It is seen from FIG. 11 that since the air velocity in the lateral direction measurement position ▴ was decreased in accordance with a decrease in the angle θ at which the air current was blown, spreading of the air current in the lateral direction was small, and since the air velocity in the lateral direction measurement position ▴ was the largest in a case where the air current V was blown against the side wall surface W1 at θ=90°, that is, a case where the air current V was blown against the side wall surface W1 at a right angle, the spreading of the air current in the lateral direction was the largest.

Next, when the air current was sent out from the blowout port of the conventional air cleaner at the angles θ=0° and 20°, the air cleaner was placed on the floor surface F so as to allow the side of the rear surface thereof to face the side wall surface W1 of the room, and the distance L from the side wall surface W1 was changed, changes in the air velocity along the ceiling surface S in a position which was 1000 mm away from the side of the front surface of the air cleaner, that is, L+1000 mm away from the side wall surface W1 were examined.

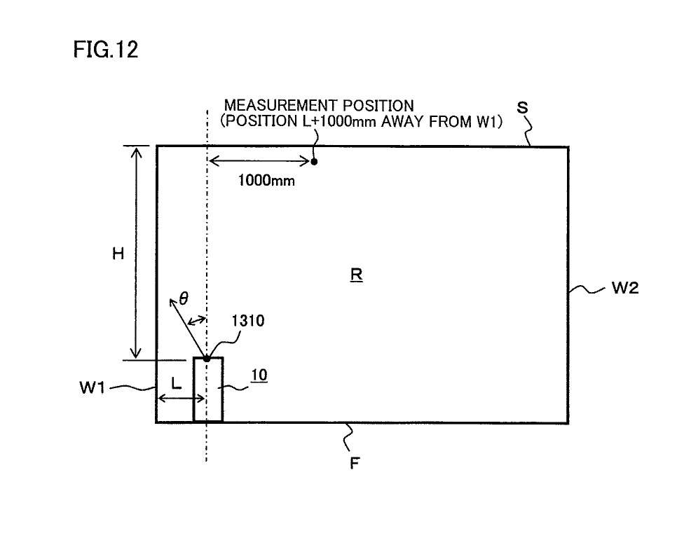

FIG. 12 is a diagram illustrating placement of the air cleaner and a measurement position where an air velocity attained by the air cleaner is measured.

As shown in FIG. 12, the conventional air cleaner 10 shown in FIG. 34 was placed in the room R as described below. The distance L from the middle position of the blowout port 1310 to the side wall surface W1 was 70 mm, 170 mm, 370 mm, 470 mm, 570 mm, or 770 mm and the distance H from the middle position of the blowout port 1310 to the ceiling surface S of the room was 1880 mm. In the conventional air cleaner 10, the angle θ formed between the direction of the flow of the air sent out from the blowout port 1310 and the vertically upward direction was 0° or 20°. When the conventional air cleaner 10 placed as described above was operated, the air velocity along the ceiling surface S was measured. The measurement position where the air velocity was measured was a position along the ceiling surface S, which was L+1000 mm away from the side wall surface W1. The air flow volume of the air sent out from the air cleaner was 4.3 m³/min.

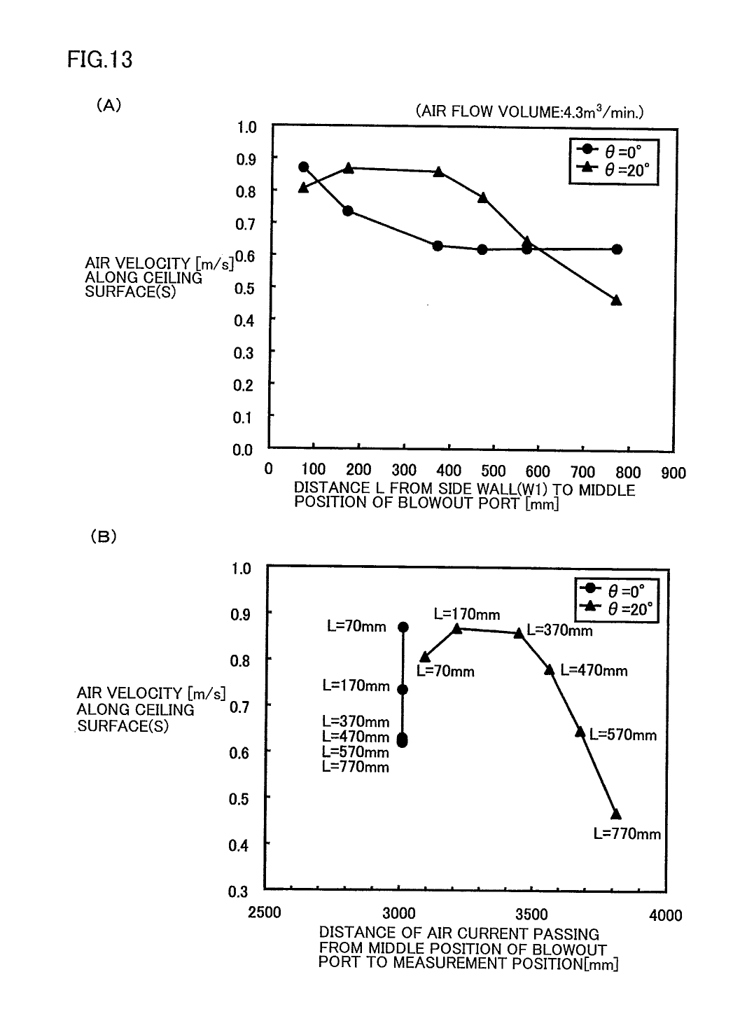

FIG. 13 is a graph showing a result of measuring the air velocity.

FIG. 13 (A) is a graph showing changes in the air velocity along the ceiling surface S in the position 1000 mm away from the side of the front surface 1100 of the air cleaner 10, that is, L+1000 mm away from the side wall surface W1 when the air current was sent out from the blowout port 1310 of the conventional air cleaner 10 at the angles θ=0° and 20°, the air cleaner 10 was placed on the floor surface F (H=1880 mm) so as to allow the side of the rear surface 1200 to face the side wall surface W1 of the room, and the distance L from the side wall surface was changed. In other words, the measurement was conducted such that not only the position where the air cleaner was placed but also the measurement position were changed in accordance with a change in L. A horizontal axis indicates a distance (unit: mm) from the side wall surface W1 to the middle position of the blowout port 1310 and a vertical axis indicates the air velocity (unit: m/s) along the ceiling surface in a position 1000 mm away from the side of the front surface of the air cleaner (L+1000 mm away from the side wall surface W1). In addition, FIG. 13 (B) is a graph showing changes in the air velocity (unit: m/s) along the ceiling surface, having a horizontal axis indicating “a distance (unit: mm), from the middle position of the blowout port 1310 to the measurement position, over which the air current passed”, instead of the horizontal axis of FIG. 13 (A), which indicates “the distance L (unit: mm) from the side wall surface W1 to the middle position of the blowout port 1310”. On the above-mentioned horizontal axis, it is seen that in the case where the air current was sent out at the angle θ=20°, the distance, from the middle position of the blowout port 1310 to the measurement position, over which the air current passed was increased in accordance with an increase in the distance L whereas in the case where the air current was sent out at the angle θ=0°, the distance, from the middle position of the blowout port 1310 to the measurement position, over which the air current passed did not change even when the distance L was changed.

In FIG. 13 (A), the case where the air current was sent out from the blowout port 1310 of the air cleaner 10 at the angle θ=0° ( in FIG. 13 (A)) and the case where the air current was sent out from the blowout port 1310 of the air cleaner 10 at the angle θ=20° (▴ in FIG. 13 (A)) were compared. In FIG. 13 (A), the air velocity in the vicinity of the ceiling surface S in the position 1000 mm away from the side of the front surface 1100 of the air cleaner 10, that is, the position L+1000 mm away from the side wall surface W1 was increased in the case of θ=0° with L<100 mm, in the case of θ=20° with 100<L<600 mm, and in the case of θ=0° with L>600 mm. As described above, it is seen from the results shown in FIG. 6, FIG. 7, and FIG. 9 that since the dust collecting performance was more enhanced in accordance with the increase in the air velocity in the vicinity of the ceiling surface S, that is, in the case of θ=20° (which corresponds to the angle in the air cleaner of the present embodiment) rather than in the case of θ=0° (which corresponds to the angle in the conventional air cleaner), the air cleaner according to the present embodiment has superiority in the dust collecting performance in a region of 100<L<600 mm.

Hereinafter, critical significance of these numerical range limitations will be described.

First, the case where the air current is sent out from the blowout port of the air cleaner at the angle θ=0° (which corresponds to the angle in the conventional air cleaner) will be described in detail. As described above, in FIG. 13 (B), the distance, from the middle position of the blowout port to the measurement position, over which the air current passes does not change in accordance with the change in the distance L. Accordingly, in a region of L>400 mm in FIG. 13 (A), the air velocity along the ceiling surface is substantially constant. However, in a region of L<400 mm in FIG. 13 (A), the air cleaner 10 and the side wall surface W1 are located in close proximity to each other and an influence of the side wall surface W1 emerges. In other words, since the air current flowing toward the rear surface of the air cleaner is decreased in accordance with an increase in the proximity of the air cleaner 10 and the side wall surface W1 (in accordance with a decrease in the distance L) and a loss is decreased, the air velocity in the measurement position is increased.

Next, the case where the air current is sent out from the blowout port of the air cleaner at the angle θ=20° (which corresponds to the angle in the air cleaner of the present embodiment) will be described in detail. In this case, since the distance, from the middle position of the blowout port to the measurement position, over which the air current passes is increased in accordance with the increase in the distance L, the air velocity in the measurement position is reduced in accordance with the increase in the distance L. However, in a region of L<170 mm in FIGS. 13 (A) and (B), the air cleaner 10 and the side wall surface W1 are located in close proximity to each other and an influence of the side wall surface W1 emerges. In other words, since the air current more collides with the side wall surface W1 in accordance with the increase in the proximity of the air cleaner 10 and the side wall surface W1 (in accordance with the decrease in the distance L) and thereby, kinetic energy is impaired and a loss is accordingly increased, the air velocity in the measurement position is decreased. In a region of 170<L<370 mm, the air velocity in the measurement position is most increased.

Next, the case of the angle θ=0° and the case of the angle θ=20° are compared.

First, when the air current is sent out from the blowout port at θ=20° in a region of L<100 mm where the air cleaner 10 and the side wall surface W1 are located in close proximity to each other and the influence of the side wall surface W1 strongly emerges, a disadvantage that the air current collides with the side wall surface W1 and thereby, the kinetic energy is impaired is increased and the air velocity in the measurement position is comparatively slow. In the case of θ=0°, due to an advantage that the air current flowing toward the rear surface of the air cleaner 10 is greatly decreased and thereby, a loss is drastically decreased, the air velocity in the measurement position is comparatively fast. Accordingly, in the region of L<100 mm, in the case of θ=0°, the air velocity in the vicinity of the ceiling surface is increased and the dust collecting performance is enhanced, as compared with the case of θ=20°.

Next, when the air current is sent out from the blowout port at θ=0° in the region of 100 mm<L<600 mm, the air current first collides with the ceiling surface S. Since the air current collides with the ceiling surface S from a vertical direction, the air current disperses in all directions. In other words, a disadvantage of the loss caused when a part of the air current flows toward the side of the rear surface of the air cleaner 10 is increased. In contrast to this, in the case of θ=20°, the air current sent out from the blowout port first collides with the side wall surface W1. Since the air current collides with the side wall surface W1 from an oblique direction and therefore, the air current hardly flows in the direction (which is a direction facing toward the floor surface F and is indicated by an arrow D in FIG. 16 mentioned later, and at this time, θ1 is an acute angle) which forms the acute angle with respect to the plane with which the air current collides, the air current converges in a direction (which is a direction facing toward the ceiling surface S and is indicated by an arrow C in FIG. 16 mentioned later and at this time, θ2 is an obtuse angle) which forms the obtuse angle with respect to the plane with which the air current collides. In other words, little air current flows toward the side of the rear surface of the air cleaner 10. This advantage is extremely great. However, although a distance over which the air current is guided toward the side of the front surface of the air cleaner 10 is elongated, as compared with the case of θ=0°, this disadvantage is small, as compared with the above-mentioned advantage. Accordingly, due to a magnitude relation of a volume lost when a part of the air current flows toward the side of the rear surface of the air cleaner 10 in the case of θ=0° and a volume lost when the distance over which the air current is guided toward the side of the front surface of the air cleaner 10 in the case of θ=20° is elongated, a difference between the air velocities along the ceiling surface S in the cases of θ=0° and θ=20° occurs. In the region of 100 mm<L<600 mm, since the loss in the case of θ=20° is small as compared with the case of θ=0°, the air velocity in the vicinity of the ceiling surface S in the case of θ=20° is increased and the dust collecting performance is enhanced. With reference to FIG. 13 (A), a difference of the air velocities in the cases of θ=20° and θ=0° becomes the largest in a case of L=370 mm (in a case where a distance between the side wall surface W1 and the rear surface of the air cleaner 10 is 300 mm).

In addition, in the region of L>600 mm, when the air current is sent out from the blowout port at θ=20°, depending on a magnitude of the distance H, there may be a case where the air current first reaches the side wall surface W1 and a case where the air current first reaches the ceiling surface S.

First, in the case where the air current first reaches the side wall surface W1, since the air current collides with the side wall surface W1 from an oblique direction, kinetic energy of the air current is hardly impaired, and since the air current hardly flows in the direction (which is a direction facing toward the floor surface F) which forms the acute angle with respect to the plane with which the air current collides, the air current converges in the direction (which is a direction facing toward the ceiling surface S) which forms the obtuse angle with respect to the plane with which the air current collides. However, since a distance over which the air current is guided toward the side of the front surface of the air cleaner 10 is elongated, the disadvantage is extremely increased.

On the other hand, in the case where the air current first reaches the ceiling surface S, the air current hardly flows in the direction (toward the side of the front surface of the air cleaner 10) which forms the acute angle with respect to the plane with which the air current collides and easily flows in the direction (toward the side of the rear surface of the air cleaner 10) which forms the obtuse angle with respect to the plane with which the air current collides. In other words, since the air current flowing toward the side of the rear surface of the air cleaner 10 is increased, the air velocity along the ceiling surface S on the side of the front surface of the air cleaner 10 is greatly impaired. On the other hand, in the case where the air current is sent out from the blowout port at θ=0°, as mentioned above, in the region of L>400 mm, the disadvantage is substantially constant, regardless of a magnitude of the distance L. Accordingly, in the region of L>600, since the disadvantage in the case of θ=0° is smaller than the disadvantage in the case of θ=20°, the air velocity in the vicinity of ceiling surface S is increased and the dust collecting performance is enhanced in the case of θ=0°.

In other words, as described above, the results shown in FIGS. 13 (A) and (B) bears out that in the air cleaner placed in the vicinity of the side wall surface W1 of the room (100 mm<L<600 mm), by employing the configuration of the air cleaner according to the present invention, the air velocity in the vicinity of the ceiling surface S is increased and the air in the room can be effectively cleaned.

In addition, FIG. 13 shows one example of the results of measuring the air velocity in a general room having H=1880 mm, that is, having a height, from the floor surface F to the ceiling surface S, of 2.5 m. Even if the distance H from the middle position of the blowout port to the ceiling surface S of the room is changed, an increase or a decrease in the distance over which the air current reaches the ceiling surface S depends on the distance H, not the angle θ at which the air current is blown out. Accordingly, in the air cleaner placed in the vicinity of the side wall surface W1 of the room (100 mm<L<600 mm), if the distance H from the middle position of the blowout port to the ceiling surface S of the room is elongated, an air flow volume is required to be accordingly increased. However, in the air cleaner according to the present invention, since the same effect as that shown in FIG. 13 can be attained, regardless of the distance from the middle position of the blowout port to the ceiling surface S of the room, the air velocity in the vicinity of the ceiling surface S is increased and the air in the room can be effectively cleaned.

Here, when an area of a floor on which the air cleaner is applied is A (m²), a height of the room is h (m), a volume of the room is V (m³), an air flow volume sent out from the air cleaner is Q (m³/min), and a dust collecting efficiency of the air cleaner is θ (%), the following equation is set forth in the Japan Electrical Manufacturers' Association Standards (JEM1467) and the floor area A is proportional to the air flow volume Q.

A=7.7×Q×θ/100

A∝Q

In addition, the volume V of the room is proportional to the floor area A and the height h of the room.

V∝A

V∝h

Accordingly, in order to effectively clean the air in the room, the air flow volume is required to be changed in accordance with the volume of the room, that is, the floor area and the height of the room.

Q∝h

In other words, in the air cleaner placed in the vicinity of the side wall surface of the room (100 mm<L<600 mm), since by changing the air flow volume Q so as to be proportional to the height of the room, that is, the distance H from the middle position of the blowout port to the ceiling surface S of the room, the same effect as that shown in FIG. 13 can be attained by the air cleaner according to the present invention, irrespective of the distance H from the middle position of the blowout port to the ceiling surface S of the room, the air velocity in the vicinity of the ceiling surface S is increased and the air in the room can be effectively cleaned.

As described above, as is seen from the results shown in FIG. 9 and FIG. 11, depending on the angle at which the air current is sent out from the blowout port of the air cleaner and the position where the air cleaner is placed, a flow of the air in the room, that is, an air velocity distribution in the vicinity of the ceiling surface S and spreading of the air current in a lateral direction, which stretches when the air current is blown against the side wall surface W1, are greatly changed. In consideration of these phenomena, a behavior of the air current in each of the air cleaner according to the present invention and the conventional air cleaner will be described.

Specifically, as in the conventional air cleaner, in the case where the air current is sent out from the blowout port of the air cleaner vertically upward (θ=0°), the air current sent out from the blowout port first collides with the ceiling surface. Since the air current collides with the ceiling surface from the vertical direction, the kinetic energy of the air current is greatly impaired and the air current disperses substantially evenly in all directions. In other words, a part of the air current flows toward the side of the rear surface of the air cleaner. Therefore, the air velocity along the ceiling surface on the side of the front surface of the air cleaner is remarkably reduced. In other words, the air current (air flow volume) flowing toward the side of the front surface of the air cleaner is impaired. In addition, when the air current collides with the ceiling surface, since the air current disperses substantially evenly in the all directions, a direction of the air current cannot be controlled and the air current cannot be sent out intentionally toward a side of a space (a living space or a space where a person is present) in which the air is desired to be cleaned.

In contrast to this, as in the air cleaner according to the present invention, in the case where the air current is sent out from the blowout port of the air cleaner obliquely backward, the air current sent out from the blowout port first collides with the side wall surface. Since the air current collides with the side wall surface from the oblique direction, the kinetic energy of the air current is hardly impaired and the air current hardly flows in the direction (which is the direction facing toward the floor surface) which forms the acute angle with respect to the plane with which the air current collides and therefore, the air current converges in the direction (which is the direction facing toward the ceiling surface) which forms the obtuse angle with respect to the plane with which the air current collides. In other words, little air current flows toward the side of the rear surface of the air cleaner. In addition, as is seen from the result shown in FIG. 11, since in a position where the air current collides with the side wall surface from the oblique direction, diffusion of the air current in the lateral direction is small, the air current hardly disperses and the air current flowing in a direction toward the ceiling surface is not much impaired. Therefore, the air velocity along the ceiling surface on the side of the front surface of the air cleaner is not much impaired though it depends on the angle of the air current sent out from the blowout port. In other words, the air current (air flow volume) flowing toward the side of the front surface of the air cleaner is not much impaired. Further, since when the air current collides with the side wall surface, the air current converges in the direction (which is the direction facing toward the ceiling surface) which forms the obtuse angle with respect to the plane with which the air current collides, the direction of the air current can be controlled. As described above, by employing the configuration of the present invention, since the distance over which the air current can reach can be drastically extended, the air current can be sent out intentionally toward the side of the space (the living space or the space where a person is present) in which the air is desired to be cleaned.

Next, in a case where by employing the configuration of the air cleaner according to the present invention, the air current flowing in the room is changed as described above, a mechanism with which the air in the room and in particular, in the position away from the air cleaner can be effectively cleaned will be described, as compared with the conventional air cleaner.

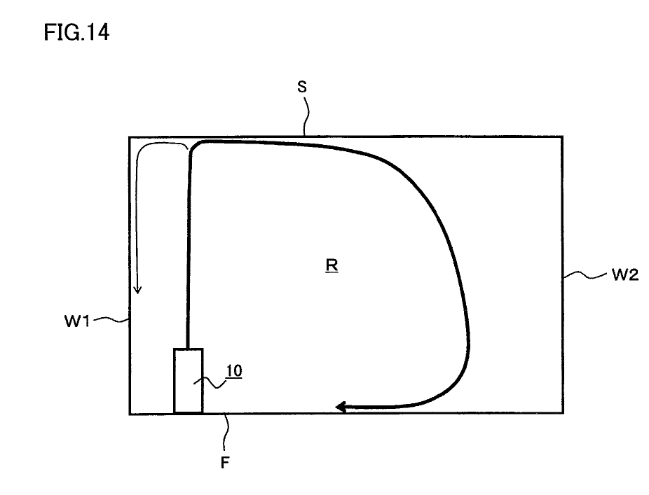

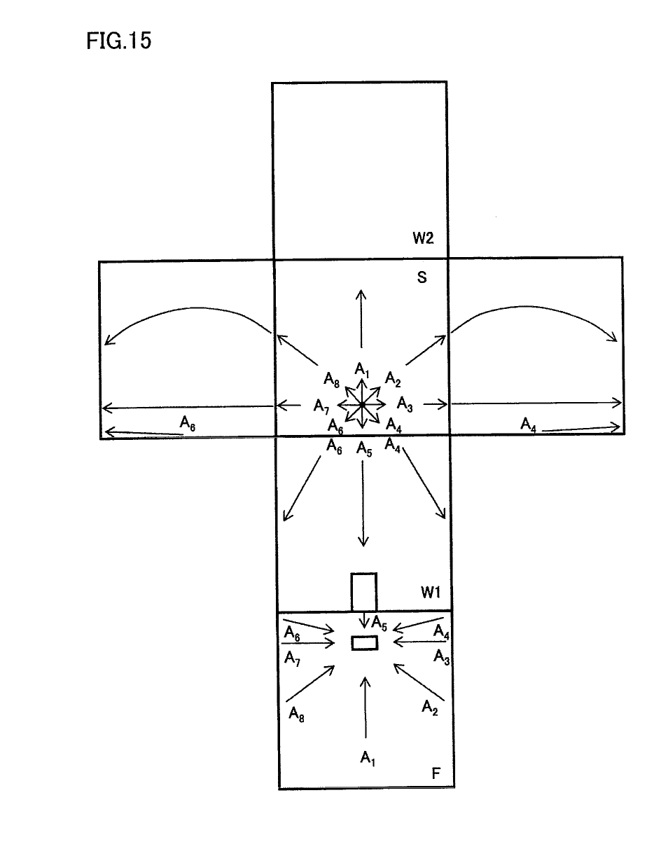

FIG. 14 is a cross-sectional view schematically illustrating an indoor air current sent out from the conventional air cleaner. FIG. 15 is a development view schematically illustrating the indoor air current (air current along the wall surface) sent out from the conventional air cleaner.

As shown in FIG. 14, cleaned air is blown out immediately upward (vertically upward) from the blowout port and the air current sent out from the conventional air cleaner 10 collides with the ceiling surface S from the vertical direction. At this time, since the air current collides with the ceiling surface S from the vertical direction, the kinetic energy is greatly impaired upon the collision and the air current disperses substantially evenly in the all directions.

Accordingly, a relationship of air currents shown in FIG. 15 is expressed by the following formula.

A₁≈A₂≈A₃≈A₄≈A₅≈A₆≈A₇≈A₈

At this time, a part of the air currents, which are A₄, A₅, and A₆ (approximately 1/4 of the whole air currents), flows toward behind the air cleaner and is immediately sucked into the air cleaner. In other words, it is difficult for the part of the air currents A₄, A₅, and A₆ (approximately ¼) to spread in the whole room, whereby the part of the air currents A₄, A₅, and A₆ hardly contributes to cleaning of the indoor air. Accordingly, since among the air currents sent out from the conventional air cleaner, the part of the air currents is uselessly sent out, the conventional air cleaner cannot effectively clean the indoor air.

When from the same viewpoint, the air cleaner according to the present invention is reviewed, the following is considered.

FIG. 16 is a cross-sectional view schematically illustrating an indoor air current sent out from the air cleaner according to the present invention. FIG. 17 is a development view schematically illustrating the indoor air current (air current along the wall surface) sent out from the air cleaner according to the present invention.

As shown in FIG. 16, cleaned air is blown out from the blowout port obliquely backward and the air current sent out from the air cleaner 1 according to the present invention first collides with the side wall surface W1 therebehind. Since the air current collides with the side wall surface W1 therebehind from the oblique direction, not the vertical direction, the kinetic energy is hardly impaired upon the collision. In addition, the air current smoothly flows along the side wall surface W1 therebehind in the direction toward the ceiling surface S (direction indicated by the arrow C in FIG. 16).

At this time, a relationship of air currents shown in FIG. 17 is expressed by the following formula.

B₁>B₂≈B₈>B₃≈B₇>B₄≈B₆>>B₅

At this time, since the air current hardly flows in the direction (which is the direction facing toward the floor surface F and is indicated by the arrow D in FIG. 16, and at this time, θ1 is an acute angle) which forms the acute angle with respect to the plane with which the air current collides, the air current flowing toward behind the air cleaner is drastically decreased, as compared with the conventional air cleaner.

In other words, the relationship of the air currents shown in FIG. 17 is expressed by the following formula.

A₅>>B₅

In other words, substantially all the air currents contribute to cleaning of the indoor air. Accordingly, it is made possible to effectively clean the indoor air by the air current sent out from the air cleaner according to the present invention.

Superiority in the effect attained by the configuration of the present invention is exhibited particularly in a position (wide space) far away from the front surface of the air cleaner. This is because the loss of the air current (air current flowing toward the side of the rear surface of the air cleaner) sent out from the conventional air cleaner is large and the loss of the air current sent out from the air cleaner according to the present invention is small. In other words, even though the air flow volumes at the blowout ports of the conventional air cleaner and the air cleaner according to the present invention are the same as each other, a difference between the air flow volumes guided toward the side of the front surfaces of the conventional air cleaner and the air cleaner according to the present invention occurs. In addition, the air current sent out from the air cleaner according to the present invention takes advantage of the Coanda effect. Since the air current is caused to travel along the wall surface from the side wall surface toward the ceiling surface, the air current hardly diffuses, and since a potential core region is extended, a distance over which the air current can reach is extended. Since the air current sent out from the conventional air cleaner hardly reaches a position far away from the air cleaner, stagnation is easily caused in a remote position where the air current does not reach and air in a region where the stagnation is caused is cleaned by diffusion based on differences among concentrations. In contrast to this, since the loss of the air current sent out from the air cleaner according to the present invention is small and the distance over which the air current can reach is extended due to the Coanda effect, the air current easily reaches the position far away from the air cleaner. Accordingly, the air current sent out from air cleaner according to the present invention exhibits an effect in the position far away from the front surface of the air cleaner and a wider space can be effectively cleaned.

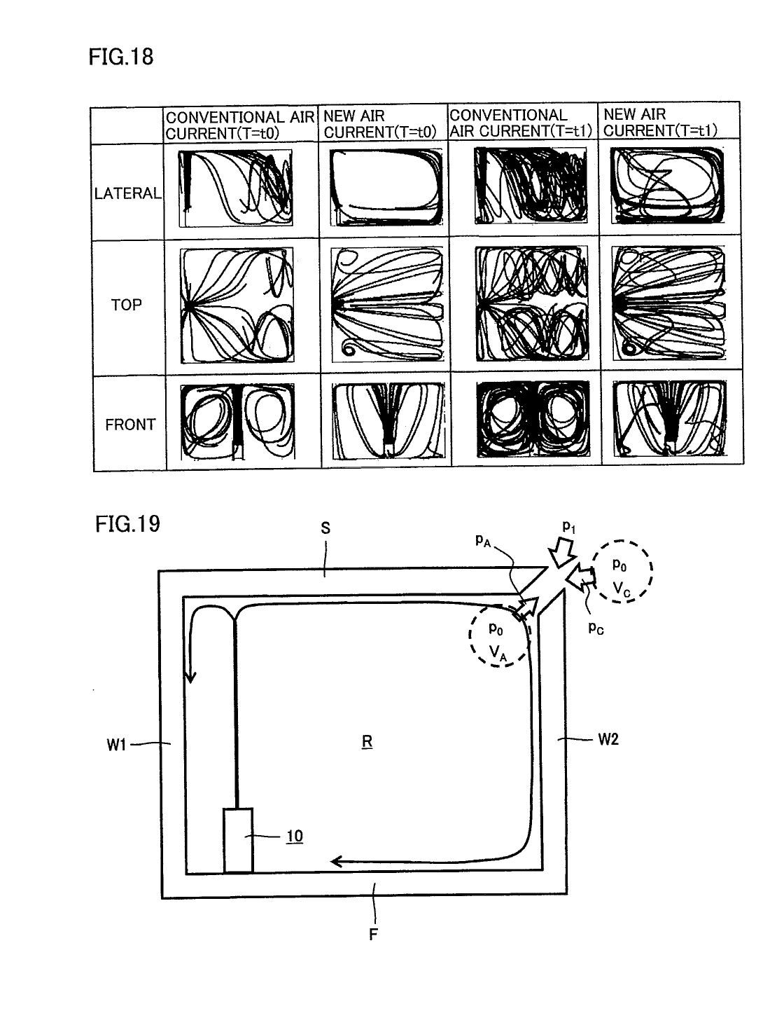

In each of the cases of the air current sent out from the conventional air cleaner and the air current sent out from the air cleaner according to the present invention, a path of the air current circulating in a space of the room, that is, a streamline was visualized by using a CFD (Computational Fluid Dynamics) analysis (computer simulation). For reference, a result thereof is shown in FIG. 18.

Conditions of the analysis are that: a room has a size of 8 tatami-mats; the same air velocity (4 m/s) at which the air current is blown out and the same air flow volume with which the air current is blown out are set for each of the cases; the air current sent out from the conventional air cleaner is set to be blown out in a vertical direction; and the air current sent out from the air cleaner according to the present invention is set such that an angle formed between the air current and the vertical direction or an angle between the air current and a side wall surface of the room is 20°. In addition, the analysis is started at a time: T=0, a time at which a streamline of a new air current (an air current sent out from the air cleaner according to the present invention) sent out from the blowout port first reaches the suction port is supposed to be T=t₀, and a time at which a streamline of a conventional air current (an air current sent out from the conventional air cleaner) sent out from the blowout port first reaches the suction port is supposed to be T=t₁. A trajectory along which a flow present at the blowout port of the air cleaner at the time: T=0 travels at each of the times: T=t₀ and T=t₁, that is, a trajectory of each of the streamlines is represented. Respective diagrams shown in an upper row in FIG. 18 correspond to the views shown in FIG. 14 and FIG. 16. Respective diagrams shown in a middle row in FIG. 18 correspond to the views of the rooms shown in FIG. 15 and FIG. 17, and in each of the respective diagrams, the ceiling surface S is rotated 90° clockwise. Respective diagrams shown in a lower row in FIG. 18 are views, in each of which the side wall surface W1 is viewed from the side of the side wall surface W2 shown in FIG. 8.

It is seen from FIG. 18 that at the time T=tₒ, the air current sent out from the air cleaner according to the present invention, which was blown out at the time: T=0, largely circulated in the room and reached again the air cleaner whereas the air current sent out from the conventional air cleaner, which was blown out at the time: T=0, was caught up in a large vortex and stagnating in a position of the room, far away from the air cleaner. In addition, for reference, a relationship between the time: T=t₁ and the time: T=t₀ is expressed by the following equation.

t₁=1.7×t₀

In other words, in the analysis, time required when the air current sent out from the conventional air cleaner largely circulates in the room and reaches again the air cleaner is 1.7 times longer than time required when the air current sent out from the air cleaner according to the present invention largely circulates in the room and reaches again the air cleaner.

In a case where a behavior of actual dust is considered, due to a condition of a mass of dust>a mass of air, movement of the dust lags behind that of the air current. In other words, the behavior of the air current shown in FIG. 18 does not represent the behavior of the dust. However, since it is considered that there is a strong correlation between a flow of the air current and the movement of the dust, these results of the CFD analysis sufficiently bear out an effect of the present invention.

In addition, as already described with reference to FIG. 7, a mechanism with which the air cleaner according to the present invention is capable of more effectively cleaning the air in the less airtight room than the conventional air cleaner will be described.

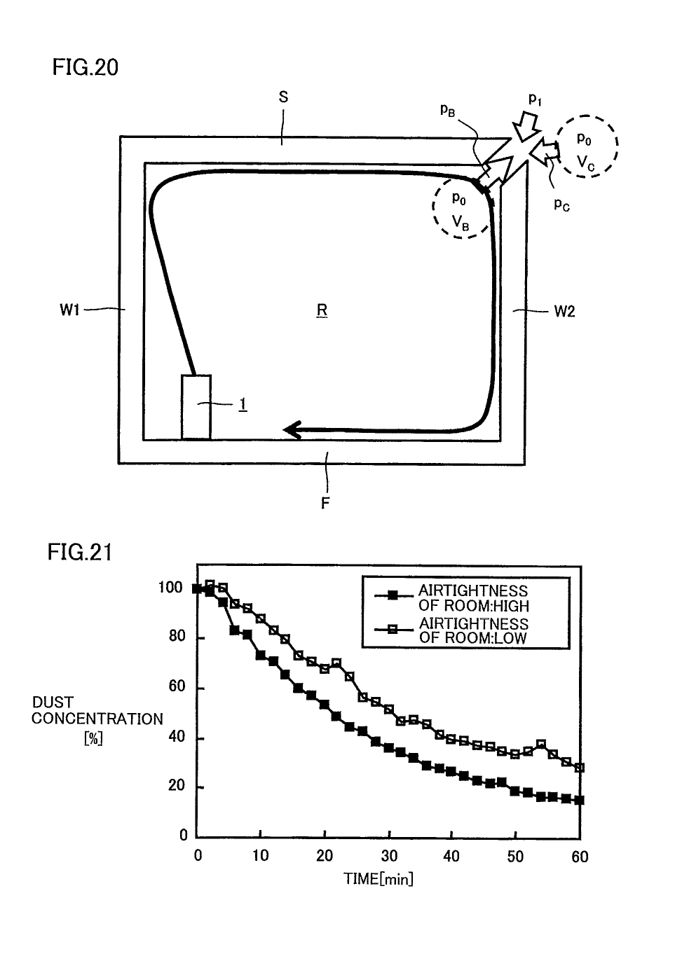

FIG. 19 is a cross-sectional view schematically illustrating the vicinities of gaps in a case where the conventional air cleaner is placed in the less airtight room (room having minute gaps in walls and windows of the room as shown in FIG. 5 (B)). In addition, FIG. 20 is a cross-sectional view schematically the vicinities of gaps in a case where the air cleaner according to the present invention is placed in the less airtight room (room having the minute gaps in the walls and the windows of the room as shown in FIG. 5 (B)).

In a case where an amount of suspended dust dispersing in the air is extremely large, for example, in a day when an amount of dispersing pollen is large, when the air cleaner is operated in a room, a difference between concentrations of the suspended dust inside and outside the room occurs. For example, in a case of the less airtight room, an amount of the suspended dust in accordance with the above mentioned difference between the concentrations enters, from the minute gaps of the walls and the windows, an inside of the room from an outside of the room.

Here, when the difference between the concentrations of the suspended dust inside and outside the room is supposed to be c (g·m⁻³), a travel distance of the suspended dust is supposed to be x (m), and a diffusion coefficient of the suspended dust is supposed to be D (m²·s⁻¹), in a case where there is no difference between pressures inside and outside the room, an amount of the suspended dust entering the inside of the room from the outside of the room per unit area and per unit time (diffusing flux) J (g·m⁻²·s⁻¹) is

$$J = - D \frac{d c}{d x} \qquad [ Equation 1 ]$$

expressed by the above Equation 1.

When an entering pressure of the suspended dust flowing into the inside of the room from the outside of the room due to the difference between the concentrations of the suspended dust outside and inside the room is supposed to be p₁, p₁ is, with a function of the difference c between the concentrations,

p₁=f(c) [Equation 2]

expressed by the above Equation 2.

Next, a pressure in the vicinity of each of the minute gaps of the walls and windows inside the room will be considered. In the case of the air current sent out from the conventional air cleaner, the pressure in the vicinity of each of the gaps is supposed to be p_{A} and a flow velocity of the air current flowing in the vicinity of each of the gaps is supposed to be V_{A}, the pressure p_{A} is, with an air density being p and an atmospheric pressure being p₀,

$${p}_{A} = {p}_{0} + \frac{1}{2} ρ {V}_{A}^{2} \qquad [ Equation 3 ]$$

expressed by the above Equation 3.

In addition, in the case of the air current sent out from the air cleaner according to the present invention, a pressure in the vicinity of each of the gaps is supposed to be p_{B} and a flow velocity of the air current flowing in the vicinity of each of the gaps is supposed to be V_{B}, the pressure p_{B} is, in the same manner as mentioned above,

$${p}_{B} = {p}_{0} + \frac{1}{2} ρ {V}_{B}^{2} \qquad [ Equation 4 ]$$

expressed by the above Equation 4.

Next, a pressure in the vicinity of each of the minute gaps of the walls and the windows outside the room will be considered.

When the pressure in the vicinity of each of the gaps is supposed to be pc and a flow velocity of the air current flowing in the vicinity of each of the gaps is supposed to be V_{C}, the pressure p_{C} is, with an air density being p and an atmospheric pressure being p₀,

$${p}_{C} = {p}_{0} + \frac{1}{2} ρ {V}_{C}^{2} \qquad [ Equation 5 ]$$

expressed by the above Equation 5.

The entering pressure of the suspended dust flowing into the room from the minute gap of each of the walls and windows is represented by the above-mentioned differential pressure. In other words, in the case of the air current sent out from the conventional air cleaner, an entering pressure of the suspended dust is supposed to be Δp_{A} and in the case of the air current sent out from the air cleaner according to the present invention, an entering pressure of the suspended dust is supposed to be Δp_{B}, the respective entering pressures are expressed as follows.

$$\begin{matrix}\begin{matrix}Δ {p}_{A} = {p}_{1} + {p}_{c} - {p}_{A} \\ = {p}_{1} + ( {p}_{0} + \frac{1}{2} ρ {V}_{C}^{2} ) - ( {p}_{0} + \frac{1}{2} ρ {V}_{A}^{2} ) \\ = {p}_{1} + \frac{1}{2} ρ ( {V}_{C}^{2} - {V}_{A}^{2} )\end{matrix} & [ Equation 6 ] \\ \begin{matrix}Δ {p}_{B} = {p}_{1} + {p}_{C} - {p}_{B} \\ = {p}_{1} + ( {p}_{0} + \frac{1}{2} ρ {V}_{C}^{2} ) - ( {p}_{0} + \frac{1}{2} ρ {V}_{B}^{2} ) \\ = {p}_{1} + \frac{1}{2} ρ ( {V}_{C}^{2} - {V}_{B}^{2} )\end{matrix} & [ Equation 7 ]\end{matrix}$$

From the result shown in FIG. 9, V_{A}<V_{B} is seen, and since V_{A} and V_{B} are expressed as functions of the square of Δp_{A} and Δ_{PB}, respectively, Δ_{PA}>>Δp_{B} results. In other words, in the case of the air current sent out from the air cleaner according to the present invention, since the air current having a comparatively high air velocity in the vicinity of the wall surface is caused to flow therealong, a dynamic pressure exerted due to the high air velocity is applied to the minute gap of each of the walls and windows and thereby, the same effect as that attained by causing the inside of the room to be under a positive pressure condition can be attained, as compared with the case of the air current sent out from the conventional air cleaner. Therefore, in the case of the air current sent out from the air cleaner according to the present invention, an amount of the suspended dust flowing into the room is decreased.

In other words, superiority of the above-mentioned effect attained by the configuration of the present invention is more exhibited particularly in the less airtight room. This is because when the air in the room is cleaned by the air cleaner, a dust concentration inside the room is lowered, as compared with that outside the room, and when the room is in such a state, diffusion (penetration pressure) due to the difference between the concentrations is caused, thereby allowing the dust to easily intrude into the room from the outside of the room. Here, in the case of the air current sent out from the conventional air cleaner, since the air velocity along the wall surface is low, an effect attained by causing a total pressure in the vicinity of the wall surface to be a positive pressure is small. In the case of the air current sent out from the air cleaner according to the present invention, since the air velocity along the wall surface is high, the effect attained by causing the total pressure in the vicinity of the wall surface to be the positive pressure is great. In other words, in the case of the air current sent out from the air cleaner according to the present invention, since due to the difference in the positive pressure effects (shield effect), it is made possible to prevent the dust from intruding into the room from the gap of the wall surface, the superior effect is more exhibited in the less airtight room.

In order to make this clear, the following analysis was conducted. Specifically, each of the conventional air cleaner (FIG. 34) and the air cleaner according to the present invention (FIG. 1) was placed and operated in the highly airtight room (FIG. 5(A)) and the less airtight room (FIG. 5(B)), and respective states in which the dust in each of the rooms was decreased were compared.

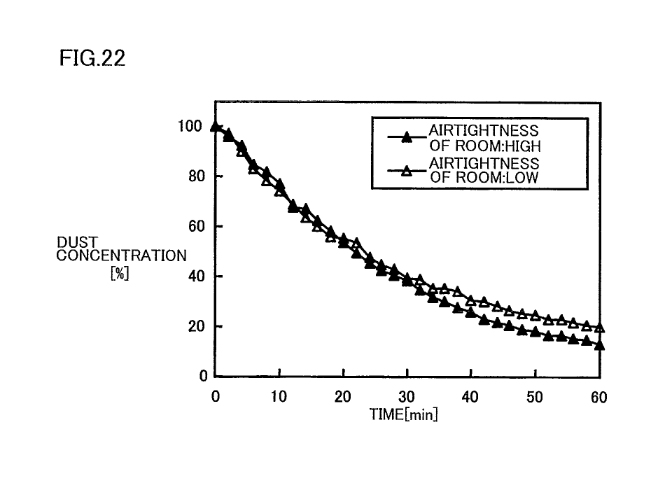

FIG. 21 is a graph showing a temporal change in a dust concentration, caused when the conventional air cleaner was operated and showing a result of comparing a case where the conventional air cleaner is placed in the highly airtight room (≡ in FIG. 21) and in a case where the conventional air cleaner is placed in the less airtight room (□ in FIG. 21). FIG. 22 is a graph showing a temporal change in a dust concentration, caused when the air cleaner according to the present invention was operated and showing a result of comparing a case where the air cleaner according to the present invention is placed in the highly airtight room (▴ in FIG. 22) and in a case where the air cleaner according to the present invention is placed in the less airtight room (Δ in FIG. 22). A horizontal axis and a vertical axis in each of FIG. 21 and FIG. 22 indicate the same as in FIG. 6.

First, it is seen from FIG. 21 that in the case of the air current sent out from the conventional air cleaner, in a case where airtightness of the room is low, a cleaning effect is remarkably impaired, as compared with a case where the airtightness is high. This is because in the case where the airtightness is low, the dust is allowed to easily intrude into the room from the outside of the room. Differences between ▪ and □ in FIG. 21 can be estimated as amounts of the dust intruding into the room from the outside of the room in the case where the airtightness of the room is low.

In contrast to this, it is seen from FIG. 22 that in the case of the air current sent out from the air cleaner according to the present invention, differences in the cleaning effect, caused due to the difference in the airtightness of the room, are small. As explained in the above description of the mechanism, this is because in the air current sent out from the air cleaner according to the present invention, since the effect attained by causing the total pressure in the vicinity of the wall surface to be the positive pressure is great, it is made possible to prevent the dust from intruding into the room from the outside of the room. Differences between ▴ and Δ in FIG. 22 can be estimated as amounts of the dust intruding into the room from the outside of the room in the case where the airtightness of the room is low.

Since the differences between ▴ and Δ in FIG. 22 were drastically decreased as compared with the differences between ▪ and □ in FIG. 21, it is seen that in the case where the airtightness of the room is low, an amount of the dust intruding into the room from the outside of the room when the air cleaner according to the present invention is operated is drastically decreased, as compared with an amount of the dust, in the case where the airtightness is low, intruding into the room from the outside of the room when the conventional air cleaner is operated.

Here, when average amounts of the dust intruding into the room from the outside of the room per unit time, which are obtained by temporally averaging the differences (the above-mentioned amounts of the dust intruding into the room from the outside of the room), caused due to the difference in the airtightness of the room, in the dust concentration are compared, the air cleaner according to the present invention can suppress the intrusion of the dust such that the average amount of the dust, per unit time, intruding into the room from the outside of the room in the case where the air cleaner according to the present invention is operated is approximately 1/30 of that in the case where the conventional air cleaner is operated. Accordingly, it is seen from the results shown in FIG. 21 and FIG. 22 and as shown in FIG. 7 that the air cleaner according to the present invention is capable of more quickly and effectively cleaning the air in the less airtight room than the conventional air cleaner.

In addition, a relationship of a size of a room and an air flow volume, which allows the air cleaner according to the present invention to more effectively cleaning the air in the room than the conventional air cleaner will be described.

In the air cleaner according to the present invention, a distance over which the air current sent out from the blowout port can reach is extended, as compared with that in the conventional air cleaner. For example, when with reference to FIG. 9, a distance from the side wall, that is, a distance over which the air current can reach when an air velocity along the ceiling surface is 0 m/s in each of the cases of the conventional air cleaner (: θ=0° in FIG. 9) and the air cleaner according to the present invention (▪: θ=20° in FIG. 9) is predicted from intersection points of the horizontal axis and the respective lines indicating the relationships between the distances from the side wall and the air velocities in FIG. 9. When the air flow volume is 4.3 m³/min, the above-mentioned distance in the case of the conventional air cleaner is approximately 3.5 m and the above-mentioned distance in the case of the air cleaner according to the present invention is approximately 4.8 m. Accordingly, it is considered that in a space with a 3.5 m or more distance from the side wall, the air cleaner according to the present invention is capable of more effectively cleaning the air in the room than the conventional air cleaner.

Here, when a floor area is supposed to be A (m²), a length of one of sides of the floor surface abutting the side wall surface on the side of the rear surface of the air cleaner is supposed to be a (m), a length of another of the sides of the floor surface is supposed to be b (m), and an air flow volume blown out from the air cleaner is supposed to be Q (m³/min), a value obtained by dividing the air flow volume Q by the floor area A is considered. Since in the room (FIG. 8) where the change in the air velocity, as shown in FIG. 9, was measured, the length of the one of the sides of the floor surface F abutting the side wall surface W1 on the side of the rear surface of the air cleaner 10 is 3.5 m, in the case of the conventional air cleaner,

$$\frac{Q}{A} = \frac{Q}{ab} = \frac{4.3}{3.5 \times 3.5} \approx 0.35 \qquad [ Equation 8 ]$$

the above-mentioned value is expressed by the above Equation 8.

Based on this, a range in which the air cleaner according to the present invention can more effectively clean the air in the room than the conventional air cleaner is expressed by the following Equation 9.

$$\frac{Q}{ab} < 0.35 \qquad [ Equation 9 ]$$

Here, it is preferable that the air cleaner is placed so as to satisfy a≦b.

In addition, when the above-mentioned value in the case of the air cleaner according to the present invention is similarly considered,

$$\frac{Q}{A} = \frac{Q}{ab} = \frac{4.3}{3.5 \times 4.8} \approx 0.25 \qquad [ Equation 10 ]$$

the above-mentioned value is expressed by the above Equation 10.

Based on this, a range in which the air cleaner according to the present invention can more effectively clean the air in the room than the conventional air cleaner is expressed by the following Equation 11.

$$0.25 ≦ \frac{Q}{ab} < 0.35 \qquad [ Equation 11 ]$$

Furthermore, by employing the configuration of the air cleaner according to the present invention, a noise can be drastically reduced, as compared with the conventional air cleaner. The configuration of the present invention allows the noise to be reduced by approximately ▴4 dB and noise energy to be reduced by approximately 60%. A mechanism which allows this drastic reduction in the noise will be described.

In the conventional air cleaner, two of the suction port and the blowout port are sources of emitting loud noises. First, the suction port will be considered. Conventionally, the suction port is arranged on the side of the front surface of the air cleaner in order to easily take the air into a main body. However, it is often the case that in order to cope with a problem of sound, to make dust accumulated in a filter less noticeable, and to improve an appearance, a panel is attached on the side of the front surface of the air cleaner so as to cover the suction port and the filter. In this case, an original purpose of making it easy to take the air into the main body by arranging the suction port on the side of the front surface of the air cleaner is greatly impaired, and since a ventilation resistance caused by the panel is large, an effect of reducing the sound is greatly impaired.

In the air cleaner according to the present invention, the suction port is arranged on the side of the rear surface, thereby preventing sound from the suction port from directly leaking to the side of the front surface. A traveling direction of the sound is caused to turn 180° before the sound has reached the side of the front surface, and during that time, the sound is attenuated due to a diffraction effect, thereby allowing the noise to be reduced. In addition, the suction port is arranged on the side of the rear surface, thereby solving a problem in an appearance, for example, by making dust accumulated in a filter less noticeable. In other words, the necessity to cover the whole filter by attaching the panel is also eliminated, thereby allowing the ventilation resistance caused by the panel to be drastically reduced and allowing the noise to be reduced.

Next, the blowout port will be considered. In the conventional air cleaner, the blowout port is arranged on a side of a top surface of the air cleaner because of consideration, for example, that no wind stress is imposed on persons and the air current is sent out in a direction in which there are no obstacles as far as possible. In addition, the blowout flow passageway communicating from the air blower to the blowout port is formed in a linear manner and sound generated in the air blower is directly emitted from the blowout port.

In the air cleaner according to the present invention, by inclining the above-mentioned blowout flow passageway, it is made unnecessary to bend the air current at a position of the blowout port of the air cleaner toward the side of the side wall surface of the room, thereby allowing a pressure loss in the air flow channel to be reduced, improving a power consumption, and allowing the noise to be reduced.

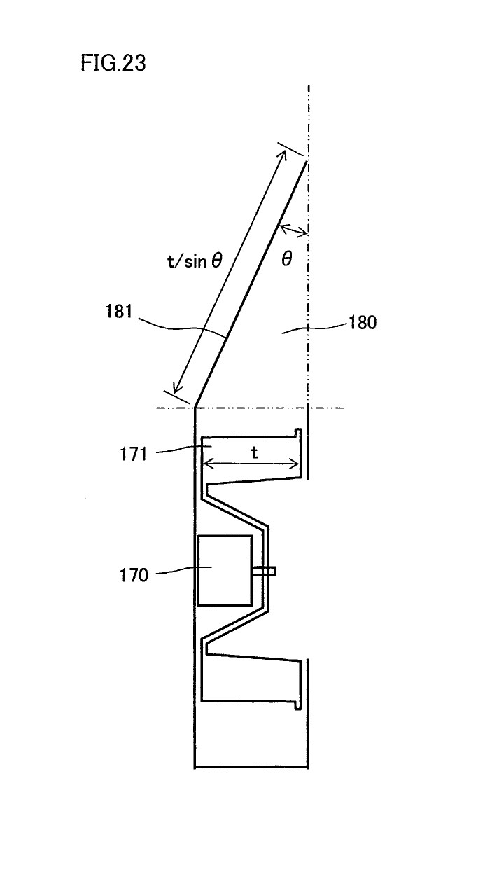

FIG. 23 is a cross-sectional view schematically illustrating a relationship between a height of the sirocco fan and a length of a front wall forming the blowout flow passageway.

As shown in FIG. 1 and FIG. 23, when the height of the sirocco fan is supposed to be t, a length, in a direction along the air current, of one wall surface among wall surfaces forming the blowout flow passageway 180 is set to be t/sin θ or more, the one wall surface arranged in a position farthest from the side wall surface W1 of the room, that is, a length of the front wall 181 is set to be t/sin θ or more. Therefore, the sirocco fan 171 is invisible from the blowout port 131, viewed from vertically above. Accordingly, before a noise generated from the air blower 170 is emitted from the blowout port 131, the noise collides with the one of the wall surfaces forming the blowout flow passageway 180 at least one time in the blowout flow passageway 180.

When sound collides with the one of the wall surfaces, the sound is reflected. However, all of the sound is not reflected but one part thereof passes through and the other thereof is attenuated when being deprived of thermal energy upon the collision with the wall surface and of vibrational energy upon vibration with the wall surface. In addition, when the sound is reflected, interference of the sound inside the blowout flow passageway 180 is easily caused and thereby, the sound is further attenuated. Further, by inclining the blowout flow passageway 180 in a direction opposite to a direction of a space where a person is present, the sound emitted from the blowout port 131 is more diffracted than in the conventional air cleaner and propagates through the space where a person is present, thereby enhancing an effect of attenuating the sound. Accordingly, the sound which has been reflected inside the inclining blowout flow passageway 180, subjected to the interference, and thereby attenuated is emitted from the blowout port 131 and is further diffracted, thereby enhancing the effect of attenuating the sound and allowing the noise to be reduced.

In addition, according to the present invention, as one example, the blowout flow passageway 180 has a cross-sectional area which gradually increases at an angle of approximately 20° (in a case where a depth dimension is constant) in accordance with an increase in proximity to a downstream part thereof, thereby suppressing exfoliation of the air current along the wall surfaces of the blowout flow passageway 180. Therefore, a flow passageway resistance is small and kinetic energy of the air current is efficiently converted to a static pressure. In other words, a part of work done by the air blower (fan), which is a static pressure rise, is covered by the static pressure generated when the kinetic energy is converted as mentioned above, whereby energy required for the work of the air blower (fan) can be reduced and the air blower (fan) can be caused to do desired work with a low power consumption.

Furthermore, according to the present invention, since the blowout flow passageway has the cross-sectional area which gradually increases at a predetermined ratio (at the angle of approximately 20° in the case where the depth dimension is constant) in accordance with the increase in the proximity to the downstream part thereof, has the depth dimension which gradually shrinks in accordance with the increase in the proximity to the downstream part thereof, and has a shape (having a cross-sectional area with a high aspect ratio) which gradually becomes narrowed in accordance with the increase in the proximity to the downstream part thereof, the aspect ratio of the cross-sectional area of the blowout port is increased and an aspect ratio of a cross section of the air current sent out from the blowout port is accordingly increased. At this time, since even if an area through which the air current flows is the same, a ratio at which the air current contacts the wall surfaces is increased, an influence of surrounding air currents is hardly exerted. Therefore, since a flow converges, a potential core of the air current is extended. This extends the distance over which the air current can reach. In other words, the above-mentioned configuration allows the kinetic energy of the air current to be converted to the static pressure and the flow velocity of the air current to be reduced, thereby enabling the extension of the distance over which such an air current can reach.

In the air cleaner according to the present invention, the air current is efficiently sent out and the distance over which the air current can reach is drastically extended, whereby not only the air in the room, which is in the space away from the air cleaner, can be more quickly cleaned but also by the positive pressure effect due to the dynamic pressure of the air current, the dust outside the room can be prevented from intruding into the room. In addition, the suction port is arranged on the side of the rear surface of the air cleaner and the blowout flow passageway is inclined backwardly upward, whereby the effects of the reflection, the interference, and the diffraction of the sound can be attained and the noise can be reduced. In other words, the air cleaner according to the present invention makes it possible to realize the low noise, to effectively clean a housing region of a user, and to obtain a comfortable space.

### Embodiment 1-2

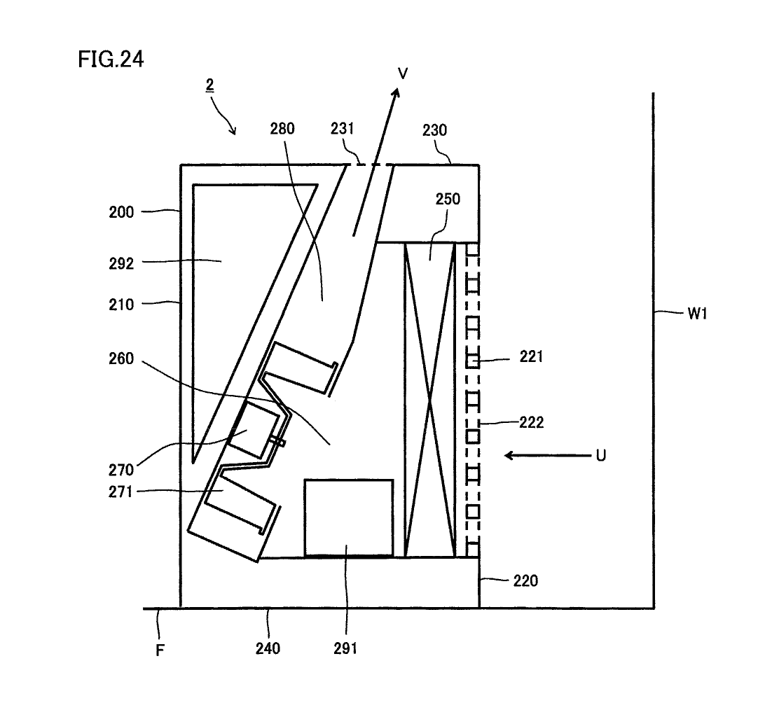

Hereinafter, an embodiment 1-2 according to the present invention will be described with reference to drawings. FIG. 24 is a side cross-sectional view schematically illustrating an air cleaner of the embodiment 1-2.

As shown in FIG. 24, the air cleaner 2 is placed on a floor surface F or a desk and used so as to be located in the vicinity of a side wall surface W1 of a room. The air cleaner 2 comprises: a main body; a suction port 222 provided in the main body and for taking in air inside the room; an air filter 250 provided in the main body as a removing part for removing dust and/or a substance present in the air taken in from the suction port 222; a blowout port 231 provided in the main body and for sending out the air treated by the air filter 250 into the room; and an air blower 270 provided in the main body as an air blowing part for moving the air from the suction port 222 to the blowout port 231.

The main body of the air cleaner 2 is held by a housing 200. The housing 200 is placed on the floor surface F and has a front surface (anterior surface) 210, a rear surface (posterior surface) 220, a top surface (upper surface) 230, and a bottom surface 240 contacting the floor surface F. The rear surface 220 of the housing 200 is covered by a perforated panel 221 in which a multitude of suction ports 222 are provided in a grid-like manner.

Here, it is supposed that a side of the air cleaner 2, from which cleaned air is sent out in a direction indicated by an arrow V is a side of the rear surface 220 and a side opposite thereto is a side of the front surface 210, with the air cleaner 2 viewed from vertically above.

The housing 200 is placed on the floor surface F so as to cause the side of the rear surface 220 to face the side wall surface W1 of the room and to be located at a distance of, for example, 300 mm from the side wall surface W1. On the top surface 230 of the housing 200, the blowout port 231 is provided. The blowout port 231 is formed so as to be of a substantially rectangular shape extending in a width direction of the housing 200 and is provided so as to face backwardly upward.

Inside the housing 200, an air flow channel which communicates from the suction port 222 to the blowout port 231 and includes a suction flow passageway 260 and a blowout flow passageway 280 is formed. Inside the air flow channel, the air blower 270 for sending out the air is placed. As a fan constituting the air blower 270, a sirocco fan 271 is used. In the air flow channel, on a downstream side of the sirocco fan 271, the blowout flow passageway 280 for flowing the air from the sirocco fan 271 to the blowout port 231 is formed. The blowout flow passageway 280 guides the air sent out by the sirocco fan 271 backwardly upward. A cross-sectional area of the blowout flow passageway 280 gradually increases in accordance with an increase in proximity to the blowout port 231 and an increase in proximity to a downstream part thereof. The blowout flow passageway 280 has the same configuration as the configuration shown in FIG. 2 and FIG. 3.

In the embodiment 1-1, the parts of the air blower 170 and the sirocco fan 171 are provided so as to stand upright whereas in the present embodiment, parts of the air blower 270 and the sirocco fan 271 are provided so as to be inclined and the air flow channel passing through the air blower 270 and the sirocco fan 271 is also inclined as similarly to the blowout flow passageway 280. In addition, in the suction flow passageway 260 between the sirocco fan 271 and the air filter 250 in the air flow channel, a humidifier 291 is provided. Further, on an upper part of the front surface side of the air cleaner 2, a water tank 292 for the humidifier 291 is provided.

In the air cleaner 2 of the present embodiment, since the air flow channel of the parts of the air blower 270 and the sirocco fan 271 is inclined as similarly to the blowout flow passageway 280, a distance between the air blower 270 and the air filter 250 is increased. Therefore, since it is made easy to take in air evenly from the whole air filter 250, a pressure loss can be reduced. In addition, in this space, the humidifier 291 may be provided. Further, in a space of the upper part of the front surface side, the water tank 292 for the humidifier 291 may be provided. Since the water tank 292 has an effect of insulating a part of sound generated in the air flow channel, a reduction in a noise is enabled.

Accordingly, since the air flow channel of the parts of the air blower 270 and the sirocco fan 271 is inclined as similarly to the blowout flow passageway 280, although an area where the air cleaner 2 is placed is slightly increased, the substantially same effect as that attained by the air cleaner 1 of the embodiment 1-1 can be attained.

### Second Embodiment

### Embodiment 2-1

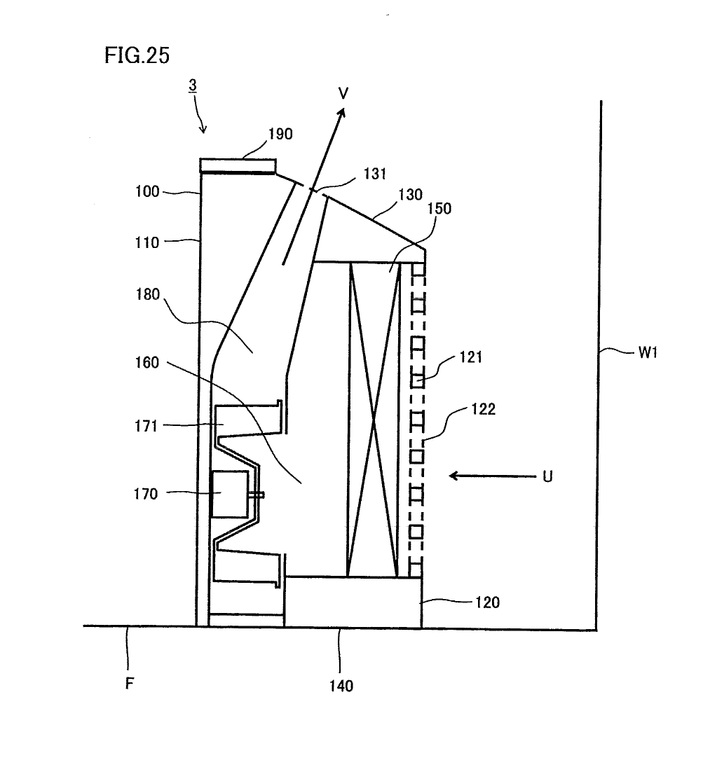

Hereinafter, an embodiment 2-1 of the present invention will be described with reference to drawings. FIG. 25 is a side cross-sectional view schematically illustrating an air cleaner of the embodiment 2-1. The same reference numerals as those used in the above descriptions of the embodiment 1-1 shown in FIG. 1 through FIG. 3 are used to denote the same parts as those in the embodiment 1-1.

As shown in FIG. 25, in the air cleaner 3 according to the present embodiment, a top surface 130 of a housing 100 is formed by a first top surface on a side of a front surface 110 on which an operation part 190 is provided and by a second top surface on a side of a rear surface 120 on which a blowout port 131 is provided. The operation part 190 is arranged on the side of the front surface 110 of the air cleaner 3 and used for switching an operation mode of the air cleaner 3. The blowout port 131 is arranged on the side of the rear surface 120 of the air cleaner 3 and used for sending out cleaned air. The other parts are the same as those of the air cleaner 1 of the embodiment 1-1. Here, it is supposed that a side of the air cleaner 3, from which cleaned air is sent out in a direction indicated by an arrow V is a side of the rear surface 120 and a side opposite thereto is a side of the front surface 110, with the air cleaner 3 viewed from vertically above.

The present embodiment allows obtainment of an effect equivalent to that attained by the air cleaner 1 of the embodiment 1-1, and a situation where the first top surface on which the operation part 190 is provided is arranged on a side facing a central part of a room in which a user is present and the second top surface on which the blowout port 131 is provided is arranged on a side facing a side wall surface W1 of the room is represented by an appearance of the air cleaner 3. In other words, this allows a user to unwittingly place the air cleaner 3 such that the blowout port 131 of the air cleaner 3 faces the side of the side wall surface W1 of the room.

In a case where the cleaned air sent out from the blowout port 131 is not caused to reach the side wall surface W1 of the room, there may be a case where an intrinsic effect of the air cleaner according to the present invention is impaired. However, since the operation part 190 of the air cleaner 3 is arranged as mentioned above, a user is allowed to unwittingly place the air cleaner 3 such that the first top surface on which the operation part 190 is arranged faces a user, thereby attaining the above-mentioned effect in an ensured manner.

Furthermore, since the operation part 190 is arranged on the side of the front surface 110 of the top surface 130 of the air cleaner 3, operability of the operation part is enhanced and an operation state of the air cleaner 3 can be easily controlled.

### Embodiment 2-2

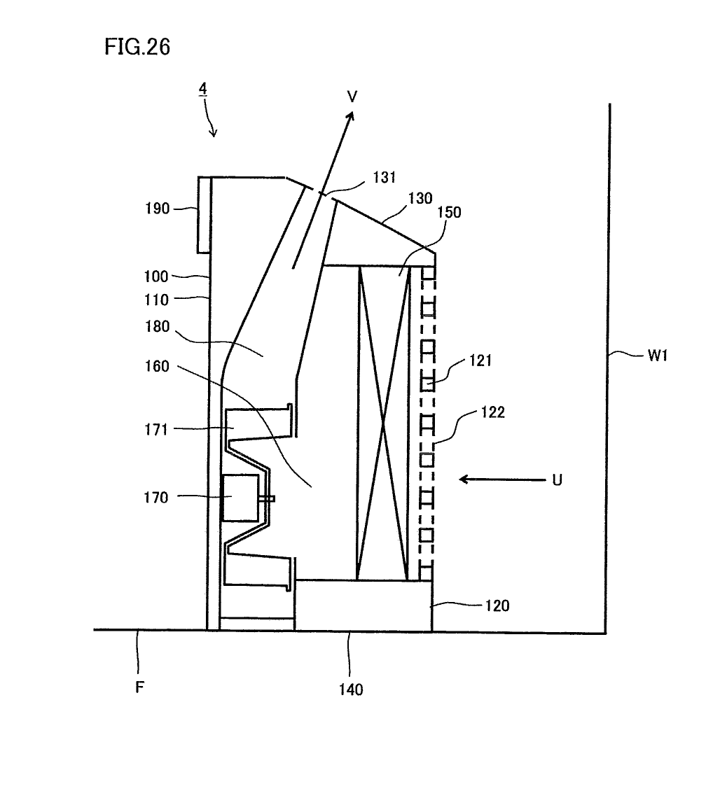

Hereinafter, an embodiment 2-2 of the present invention will be described with reference to drawings. FIG. 26 is a side cross-sectional view schematically illustrating an air cleaner of the embodiment 2-2. The same reference numerals as those used in the above descriptions of the embodiment 1-1 shown in FIG. 1 through FIG. 3 are used to denote the same parts as those in the embodiment 1-1.

As shown in FIG. 26, in the air cleaner 4 of the present embodiment, an operation part 190 is provided on an upper part of a side of a front surface 110 of the air cleaner 4. The other parts are the same as those of the air cleaner 1 of the embodiment 1-1. Here, it is supposed that a side of the air cleaner 4, from which cleaned air is sent out in a direction indicated by an arrow V is a side of the rear surface 120 and a side opposite thereto is a side of the front surface 110, with the air cleaner 4 viewed from vertically above.

The air cleaner 4 of the present embodiment allows obtainment of an effect equivalent to that attained by the air cleaner 1 of the embodiment 1-1, and since the operation part 190 is provided on the upper part of the side of the front surface 110 of the air cleaner 4, viewability of the operation part 190 is enhanced and an operation state of the air cleaner 4 can be easily checked.

Accordingly, the present embodiment allows the obtainment of an effect equivalent to that attained by the air cleaner of the embodiment 2-1, though operability is slightly inferior, as compared with the embodiment 2-1.

### Embodiment 2-3

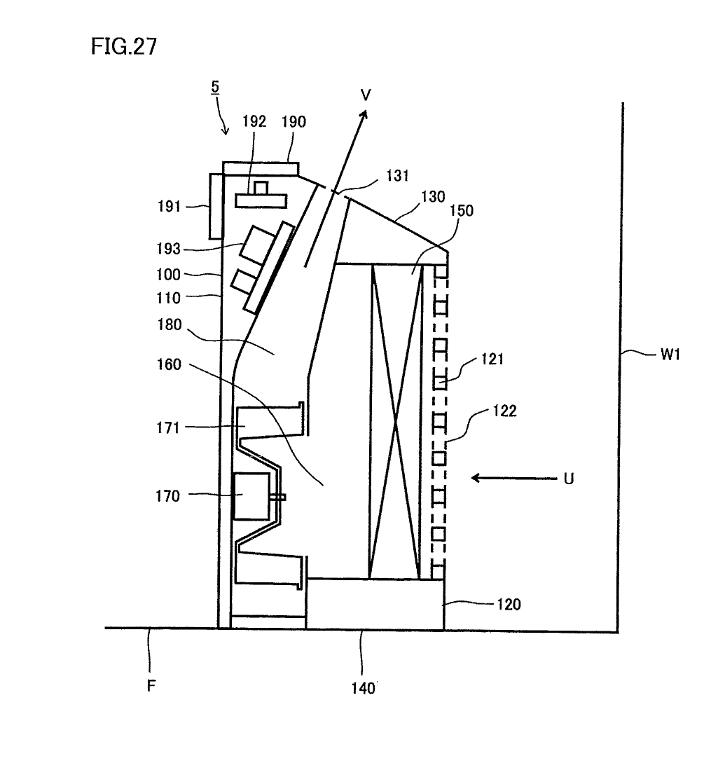

Hereinafter, an embodiment 2-3 of the present invention will be described with reference to drawings. FIG. 27 is a side cross-sectional view schematically illustrating an air cleaner of the embodiment 2-3. The same reference numerals as those used in the above descriptions of the embodiment 1-1 shown in FIG. 1 through FIG. 3 are used to denote the same parts as those in the embodiment 1-1.

As shown in FIG. 27, in the air cleaner 5 of the present embodiment, a display part 191 is provided on an upper part of a side of a front surface 110 of the air cleaner 5, and in a space surrounded by three planes of a top surface 130 of a housing 100, the front surface 110 of the housing 100, and a backwardly upward inclination part 181d (FIG. 2) of a blowout flow passageway 180, a control board 192 for an operation part 190 and for the display part 191 as well as a power source board 193 for the operation part 190 and for the display part 191 are arranged. The other parts are the same as those of the air cleaner 1 of the embodiment 1-1. Here, it is supposed that a side of the air cleaner 5, from which cleaned air is sent out in a direction indicated by an arrow V is a side of the rear surface 120 and a side opposite thereto is a side of the front surface 110, with the air cleaner 5 viewed from vertically above.

The air cleaner 5 of the present embodiment allows obtainment of an effect equivalent to that attained by the air cleaner 1 of the embodiment 1-1, and since the operation part 190 is provided on a side of the front surface 110 of the top surface 130 of the air cleaner 5, operability of the operation part is enhanced and an operation state of the air cleaner can be easily controlled, and further, since the display part 191 is provided on the upper part of the side of the front surface 110 of the air cleaner 5, viewability of the display part is enhanced and the operation state of the air cleaner 5 can be easily checked. In addition, since in the above-mentioned space, the control board 192 and the power source board 193 can be housed and the power source board 193 can be arranged in a position in close proximity to the control board 192, a length of a cord (not shown) for electrically connecting the control board 192 and the power source board 193 can be shortened.

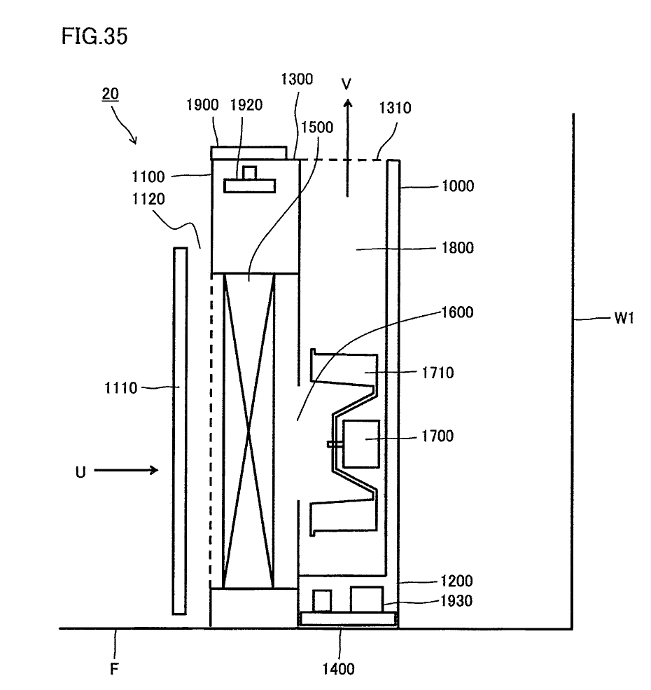

As shown in FIG. 35, in the conventional air cleaner 20, since a space where the control board 1920 is arranged is narrow, for example, there may be a case where the control board 1920 is arranged in a space in front of the air flow channel and above the air filter 1500 and the power source board 1930 is arranged in a bottom part of the housing 1000 and below the air blower 1700, thereby arranging the two boards in two separate positions. In such a case, a long cord (not shown) for electrically connecting the control board 1920 and the power source board 1930 is required. In this case, if no attention regarding methods of routing and fixing such a comparatively long cord is paid, for example, there may be a case where a failure that upon manufacturing and assembling of the air cleaner or upon repairing of the air cleaner, the cord is caught by other members and damaged occurs or a case where for example, when the housing vibrates along with rotations of the air blower, the cord contacts the housing, thereby causing an extremely harsh noise of clattering sound. Therefore, the cord is required to be wrapped by a vibration-proof material or to be bundled and fixed, thereby causing inconvenience that the number of parts and the number of processes are increased.

In the air cleaner 5 according to the present invention, as mentioned above, since the length of the cord (not shown) can be shortened, the failure, occurring upon manufacturing and assembling, that the cord is caught and the problem of the noise caused in the case of the conventional air cleaner can be obviated, the number of the parts and the number of the processes can be reduced, and a low-cost air cleaner can be obtained.

The second embodiment is summed up as follows.

(1) The air cleaner is placed on the floor or the desk and used so as to be located in the vicinity of the side wall surface of the room, comprising: the main body; the suction port provided in the main body and for taking in the air inside the room; the removing part provided in the main body and for removing dust and/or a substance present in the air taken in from the suction port; the blowout port provided in the main body and for sending out the air treated by the removing part into the room; and the air blowing part provided in the main body and for moving the air from the suction port to the blowout port. When the distance from the middle position of the blowout port to the side wall surface of the room is supposed to be L [mm], the distance from the middle position of the blowout port to the ceiling surface of the room is supposed to be H [mm], and the air cleaner is placed and used in the position which allows the distance L to be the value selected from among the values in the range of 100<L<600, in order to cause the air sent out from the blowout port to first reach the side wall surface of the room, the angle θ[°] formed between the direction in which the air is sent out from the blowout port and the vertically upward direction is set to be in the range of tan⁻¹(L/H)<≦35.

The air cleaner configured as mentioned above is characterized in that the top surface of the housing of the air cleaner is arranged so as to have the two substantially planar surfaces, which are divided and different, of the first top surface, arranged on the one side thereof, on which the operation part for switching the operation mode of the air cleaner is provided and the second top surface, arranged on the other thereof, on which the blowout port for sending out the cleaned air is provided, and with the air cleaner viewed from vertically above, the first top surface is arranged on the side which is different from the side from which the cleaned air is sent out, with respect to the second top surface.

By employing this configuration, the situation where the first top surface on which the operation part is provided is arranged on the side facing the central part of the room in which a user is present and the second top surface on which the blowout port is provided is arranged on the side facing the side wall surface of the room is represented by the appearance of the air cleaner. The operation part of the air cleaner is arranged as mentioned above, thereby allowing a user to unwittingly place the air cleaner such that the blowout port of the air cleaner faces the side of the side wall surface of the room.

Since this allows the arrangement of the second top surface, on which the blowout port is provided, on the side of the side wall surface of the room, an amount of the air current which is sent out from the blowout port and immediately sucked into the air cleaner is decreased and spreading of the air current, in the lateral direction, which is sent out from the blowout port, is suppressed, thereby drastically extending the distance over which the air current can reach and drastically increasing the air velocity in the position in the room, which is away from the air cleaner. As a result, an effect of improving an air environment in the position in the room, away from the air cleaner, can be enhanced and an indoor air in a wide range can be effectively cleaned. In addition, since the air current uselessly sent out can be decreased, even when an amount of the air flow volume is small, a desired effect of improving the air environment can be attained. Since the same effect of improving the air environment can be realized even with the small amount of the air flow volume, the noise is reduced. In addition, in a case where gaps are present in windows and others provided in the wall surfaces and the airtightness of the room is low, an air velocity in the vicinity of the wall surface of the room can be increased, thereby attaining a positive pressure effect and allowing a reduction in an amount of dust intruding into the room from the outside of the room.

In the case where the cleaned air sent out from the blowout port is not caused to reach the side wall surface of the room, there may be a case where the above-mentioned effect of the present air cleaner is impaired. However, since the operation part of the air cleaner is arranged as mentioned above, a user is allowed to unwittingly place the air cleaner such that the first top surface on which the operation part is arranged faces a user, thereby attaining the above-mentioned effect in an ensured manner.

Furthermore, the operation part is arranged as mentioned above, thereby allowing an easy-to-operate air cleaner to be obtained.

(2) The air cleaner configured as mentioned above is characterized in that a sloped plane which is gradually inclined downward in accordance with a decrease in proximity to the first top surface constitutes the second top surface.

By employing this configuration, the blowout port comes to be arranged so as to face the side wall surface.

As a path along which a noise emitted from the blowout port arranged as mentioned above propagates to a central side of the room where a user is present, there are a path along which after colliding with the side wall surface and being reflected, the noise propagates thereto and a path along which the noise propagates thereto directly from the blowout port. Since the noise propagating along the former path is reflected and absorbed by the side wall surface and is attenuated while propagating, such a noise can be reduced. In addition, although the noise propagating along the latter path is diffracted while propagating, since the blowout port is inclined so as to face the side wall surface, an effect of the diffraction is enhanced and the noise is attenuated while propagating, thereby allowing the reduction in the noise.

(3) The air cleaner configured as mentioned above is characterized in that a sloped plane which is gradually inclined downward in accordance with a decrease in proximity to the second top surface constitutes the first top surface.

By employing this configuration, the first top surface on which the operation part is provided comes to be arranged so as to face the central side of the room where a user is present. This enhances viewability of the operation part and an operation state of the air cleaner can be easily checked.

(4) The air cleaner is placed on the floor or the desk and used so as to be located in the vicinity of the side wall surface of the room and comprises: the main body; the suction port provided in the main body and for taking in the air inside the room; the removing part provided in the main body and for removing dust and/or a substance present in the air taken in from the suction port; the blowout port provided in the main body and for sending out the air treated by the removing part into the room; and the air blowing part provided in the main body and for moving the air from the suction port to the blowout port. When the distance from the middle position of the blowout port to the side wall surface of the room is supposed to be L [mm], the distance from the middle position of the blowout port to the ceiling surface of the room is supposed to be H [mm], and the air cleaner is placed and used in the position which allows the distance L to be the value selected from among the values in the range of 100<L<600, in order to cause the air sent out from the blowout port to first reach the side wall surface of the room, the angle θ[°] formed between the direction in which the air is sent out from the blowout port and the vertically upward direction is set to be in the range of tan⁻¹(L/H)<θ≦35.

The air cleaner configured as mentioned above is characterized in that the blowout port for sending out the cleaned air is provided on the top surface of the housing of the air cleaner, the operation part for switching the operation mode of the air cleaner is provided on the upper part of the side of the housing of the air cleaner, and with the air cleaner viewed from vertically above, the operation part is provided on the upper part of the side of the housing on the side which is different from the side, from which the cleaned air is sent out, with respect to top surface on which the blowout port is provided.

By employing this configuration, a situation where the side of the housing of the air cleaner, on which the operation part is provided is arranged in a central side of the room where a user is present is represented by an appearance of the air cleaner. The operation part of the air cleaner is arranged as mentioned above, thereby allowing a user to unwittingly place the air cleaner such that the air current blown out is sent out to the side of the side wall surface of the room.

Therefore, an amount of the air current which is sent out from the blowout port and immediately sucked into the air cleaner is decreased and spreading of the air current, in the lateral direction, which is sent out from the blowout port, is suppressed, thereby drastically extending the distance over which the air current can reach and drastically increasing the air velocity in the position in the room, which is away from the air cleaner. As a result, an effect of improving an air environment in the position in the room, away from the air cleaner, can be enhanced and an indoor air in a wide range can be effectively cleaned. In addition, since the air current uselessly sent out can be decreased, even when an amount of an air flow volume is small, a desired effect of improving the air environment can be attained. Since the same effect of improving the air environment can be realized even with the small amount of the air flow volume, the noise is reduced. In addition, in a case where gaps are present in windows and others provided in the wall surface and the airtightness of the room is low, an air velocity in the vicinity of the wall surface of the room can be increased, thereby attaining a positive pressure effect and allowing a reduction in an amount of dust intruding into the room from the outside of the room.

In the case where the cleaned air sent out from the blowout port is not caused to reach the side wall surface of the room, there may be a case where the above-mentioned effect of the present air cleaner is impaired. However, since the operation part of the air cleaner is arranged as mentioned above, a user is allowed to unwittingly place the air cleaner such that the upper part of the side of the housing on which the operation part is arranged faces a user, thereby attaining the above-mentioned effect in an ensured manner.

Furthermore, by arranging the operation part as mentioned above, the air cleaner whose operation part is easily viewable from a remote position can be obtained.

(5) The air cleaner configured as mentioned above is characterized in that a sloped plane which is gradually inclined downward in accordance with a decrease in proximity to the upper part of the side of the housing on which the operation part is provided constitutes the top surface.

By employing this configuration, the blowout port comes to be arranged so as to face the side wall surface of the room.

As a path along which a noise emitted from the blowout port arranged as mentioned above propagates to a central side of the room where a user is present, there are a path along which after colliding with the side wall surface and being reflected, the noise propagates thereto and a path along which the noise propagates thereto directly from the blowout port. Since the noise propagating along the former path is reflected and absorbed by the side wall surface and is attenuated while propagating, such a noise can be reduced. In addition, although the noise propagating along the latter path is diffracted while propagating, since the blowout port is inclined so as to face the side wall surface, an effect of the diffraction is enhanced and the noise is attenuated while propagating, thereby allowing the reduction in the noise.

(6) The air cleaner configured as mentioned above is characterized in that the air cleaner further comprises the display part and with the air cleaner viewed from vertically above, the display part is provided on the upper part of the side of the housing on the side which is different from the side from which the cleaned air is sent out, with respect to the top surface on which the blowout port is provided.

By employing this configuration, a situation where the side of the housing of the air cleaner, on which the display part is provided, is arranged on a central side of the room where a user is present is represented by an appearance of the air cleaner.

Since the display part of the air cleaner is arranged as mentioned above, a user is allowed to unwittingly place the air cleaner such that the blowout port faces the side of the side wall surface of the room. This allows the above-mentioned effect to be attained in an ensured manner.

Furthermore, by arranging the display part as mentioned above, the air cleaner whose display part is easily viewable from a remote position can be obtained.

(7) The air cleaner configured as mentioned above is characterized in that the blowout port is provided on the top surface of the housing and in the position in the vicinity to the side wall surface of the room; the air blower is provided in a lower part inside the housing and in the position away from the side wall surface of the room such that an outlet of the air blower faces upward; the blowout flow passageway in which the outlet of the air blower and the blowout port communicate with each other is configured so as to allow communication from the outlet of the air blower, which is provided on a upper part inside the housing and in the position away from the side wall surface of the room, to the blowout port, which is provided on the top surface of the housing and in the vicinity to side wall surface of the room and so as to be inclined from the outlet of the air blower toward the blowout port; and in the space surrounded by the three planes of the top surface, the side of the housing on a side which is different from the side from which cleaned air is sent out from the blowout port, with the air cleaner viewed from vertically above, and the side wall of the blowout flow passageway, on a side which is different from the side from which the cleaned air is sent out from the blowout port, with the air cleaner viewed from vertically above, the control board for the operation part and/or for the display part as well as the power source board for the operation part and/or for the display part are arranged.

By employing this configuration, the space is produced in the part surrounded by the above-mentioned three planes, and in this part, the control board for the operation part and/or for the display part as well as the power source board for the operation part and/or for the display part are arranged.

This allows the power source board and the control board to be arranged in the vicinity to each other, thereby allowing a length of a cord for electrically connecting the control board and the power source board to be shortened.

### Third Embodiment

### Embodiment 3-1

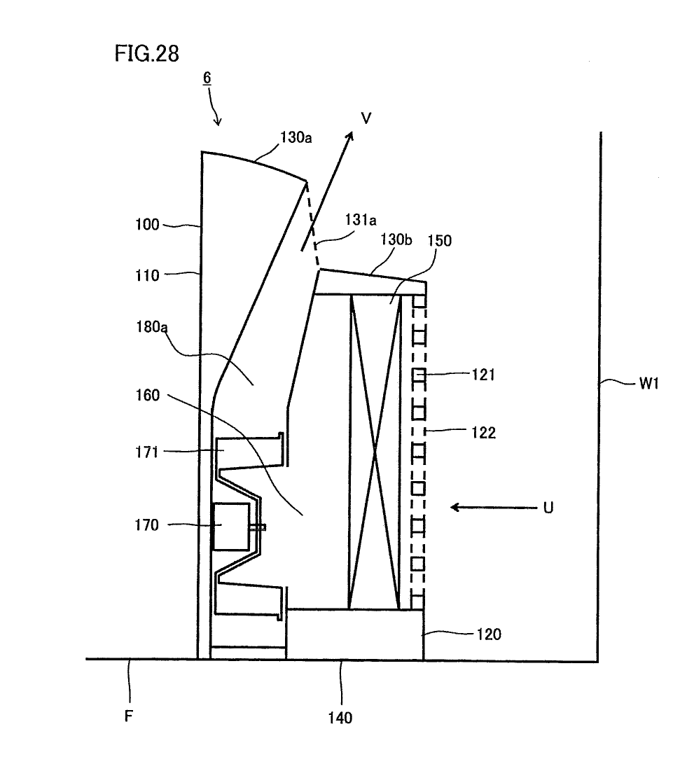

Hereinafter, an Embodiment 3-1 of the Present Invention Will be described with reference to drawings. FIG. 28 is a side cross-sectional view schematically illustrating an air cleaner of the embodiment 3-1. The same reference numerals as those used in the above descriptions of the embodiment 1-1 shown in FIG. 1 through FIG. 3 are used to denote the same parts as those in the embodiment 1-1.

As shown in FIG. 28, in the air cleaner 6 of the present embodiment, two of an upper top surface part 130a and a lower top surface part 130b constitute a top surface of a housing 100 via a split-level part, the blowout port 131a is arranged in the above-mentioned split-level part so as to face a side wall surface W1 of a room. A blowout flow passageway 180a is formed so as to allow communication between a blowout port 131a and an outlet of an air blower 170. The other parts are the same as those of the air cleaner 1 of the embodiment 1-1.

In the air cleaner 6 of the present embodiment, sound emitted from the blowout port 131a does not directly propagate into a housing region where a user is present but propagates through diffraction. Energy of the sound propagating from the blowout port 131a through the diffraction is far attenuated, as compared with that of the sound directly propagating into the housing region where a user is present, thereby drastically decreasing the sound propagating from the blowout port 131a into the housing region where a user is present. This action causes the sound emitted from the blowout port 131a to be drastically attenuated due to the diffraction effect and to propagate into the housing region, thereby allowing a drastic reduction in a noise emitted from the blowout port 131a of the air cleaner 6.

Accordingly, the blowout port 131a is provided so as to face the side wall surface W1, though a dimension of the air cleaner 6 in a height direction becomes slightly large, thereby allowing an effect of further reducing the noise to be attained, as compared with the air cleaner of the first embodiment.

### Embodiment 3-2

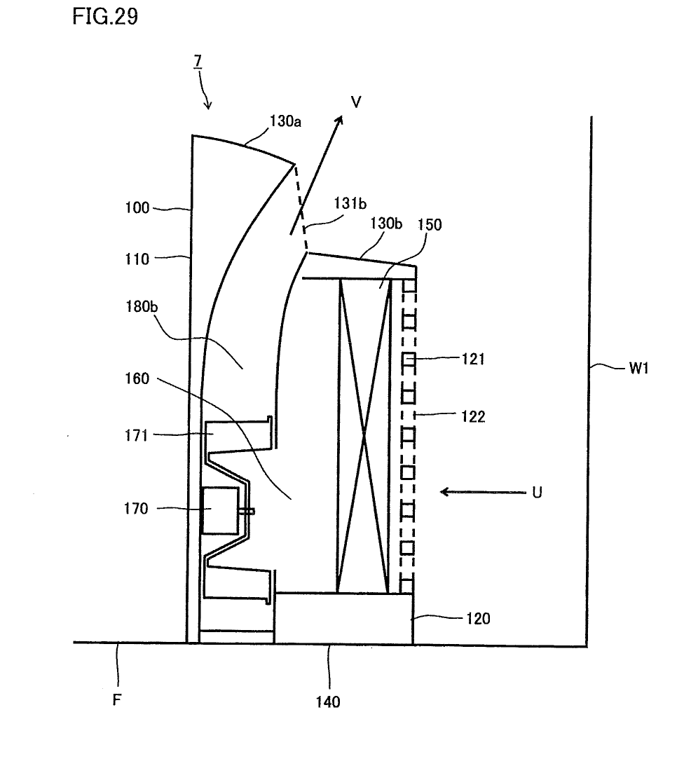

Hereinafter, an embodiment 3-2 of the present invention will be described with reference to drawings. FIG. 29 is a side cross-sectional view schematically illustrating an air cleaner of the embodiment 3-2. The same reference numerals as those used in the above descriptions of the embodiment 1-1 shown in FIG. 1 through FIG. 3 are used to denote the same parts as those in the embodiment 1-1.

As shown in FIG. 29, in the air cleaner 7 of the present embodiment, two of an upper top surface part 130a and a lower top surface part 130b constitute a top surface of a housing 100 via a split-level part, the blowout port 131b is arranged in the above-mentioned split-level part so as to face a side wall surface W1 of a room. A blowout flow passageway 180b is formed so as to allow communication between a blowout port 131b and an outlet of an air blower 170. In particular, in the air cleaner 7 of the present embodiment, the blowout flow passageway 180b is curved toward a side of the side wall surface W1 of the room and is arranged such that the blowout port 131b faces the side wall surface W1. The other parts are the same as those of the air cleaner 1 of the embodiment 1-1.

In the air cleaner 7 of the present embodiment, the blowout flow passageway 180b is smoothly curved so as to allow connection from the outlet of the air blower 170 to the blowout port 131b, thereby allowing suppression of an increase in a flow passageway resistance.

Furthermore, sound emitted from the blowout port 131b does not directly propagate into a housing region where a user is present but propagates through diffraction. Energy of the sound propagating from the blowout port 131b through the diffraction is far attenuated, as compared with that of the sound directly propagating into the housing region where a user is present, thereby drastically decreasing the sound propagating from the blowout port 131b into the housing region where a user is present. This action causes the sound emitted from the blowout port 131b to be drastically attenuated due to the diffraction effect and to propagate into the housing region, thereby allowing a drastic reduction in a noise emitted from the blowout port 131b.

Accordingly, the blowout port 131b is provided so as to face the side wall surface W1, though a dimension of the air cleaner 7 in a height direction becomes slightly large, thereby suppressing the increase in the flow passageway resistance and allowing an effect of further reducing the noise to be attained, as compared with the air cleaner of the first embodiment.

The third embodiment is summed up as follows.

(1) The air cleaner is placed on the floor or the desk and used so as to be located in the vicinity of the side wall surface of the room and comprises: the main body; the suction port provided in the main body and for taking in the air inside the room; the removing part provided in the main body and for removing dust and/or a substance present in the air taken in from the suction port; the blowout port provided in the main body and for sending out the air treated by the removing part into the room; the air blowing part provided in the main body and for moving the air from the suction port to the blowout port; and the blowout flow passageway for flowing the air from this air blowing part to the blowout port.

The air cleaner configured as mentioned above is characterized in that the blowout flow passageway is curved and/or inclined toward the side of the side wall surface of the room.

In this configuration, sound emitted from the blowout port is released into the side of the side wall surface of the room. Since sound waves has fine straight traveling properties, in order for the emitted sound to propagate into the housing region, the sound comes to be reflected by the side wall surface of the room and propagate or comes to propagate from the blowout port through diffraction. Therefore, the sound propagating directly from the blowout port into the housing region where a user is present is drastically decreased. Further, also inside the blowout flow passageway, the sound is reflected by wall surfaces.

This allows attenuation of one part of the sound reflected by the side wall surface of the room, caused when energy of the one part of the sound is absorbed by the side wall surface of the room and allows attenuation of the other part of the sound reflected by the side wall surface, caused due to interference upon the reflection. In addition, energy of the sound propagating from the blowout port through the diffraction is far attenuated, as compared with that of the sound directly propagating into the housing region where a user is present. Further, also inside the blowout flow passageway, since the sound is reflected by the wall surfaces, the attenuation of the one part of the sound, caused when the energy of the sound is absorbed by the wall surfaces, is allowed and the attenuation of the energy of the other part of the sound, due to the interference upon the reflection, is allowed. In other words, the sound emitted from the blowout port is drastically attenuated by the interference effect due to the reflection and by the diffraction effect and thereafter, propagates into the housing region, thereby allowing the drastic reduction in the noise emitted from the blowout port of the air cleaner.

(2) The air cleaner configured as mentioned above is characterized in that the blowout flow passageway is curved and/or inclined at an angle in a range of 10° through 35°.

This configuration allows suppression of an increase in a flow passageway resistance in a curved portion and/or an inclined portion of the blowout flow passageway.

Due to this, although the more the blowout flow passageway is curved and/or inclined, the more the effects of the reflection and the interference are enhanced, the flow passageway resistance is greatly increased and the effect of reducing the noise is impaired. Therefore, by setting the angle, at which the blowout flow passageway is curved and/or inclined, in the range of 10° through 35°, the effects of the reflection and the interference of the sound are attained while the increase in the flow passageway resistance is suppressed, thereby enhancing the effect of reducing the noise.

(3) The air cleaner configured as mentioned above is characterized in that the blowout port is provided so as to face the side wall surface of the room.

In this configuration, the sound emitted from the blowout port is released into the side of the side wall surface of the room. Since sound waves has fine straight traveling properties, in order for the emitted sound to propagate into the housing region, the sound comes to be reflected by the side wall surface of the room and to propagate or to propagate from the blowout port through diffraction, thereby drastically decreasing the sound propagating directly from the blowout port into the housing region where a user is present.

Due to this, the sound emitted from the blowout port does not directly propagate into the housing region where a user is present but propagates through the diffraction. The energy of the sound propagating from the blowout port through the diffraction is far attenuated, as compared with that of the sound directly propagating into the housing region where a user is present, thereby drastically decreasing the sound propagating from the blowout port into the housing region where a user is present. This action causes the sound emitted from the blowout port to be drastically attenuated due to the diffraction effect and to propagate into the housing region, thereby allowing the drastic reduction in the noise emitted from the blowout port.

### Fourth Embodiment

### Embodiment 4-1

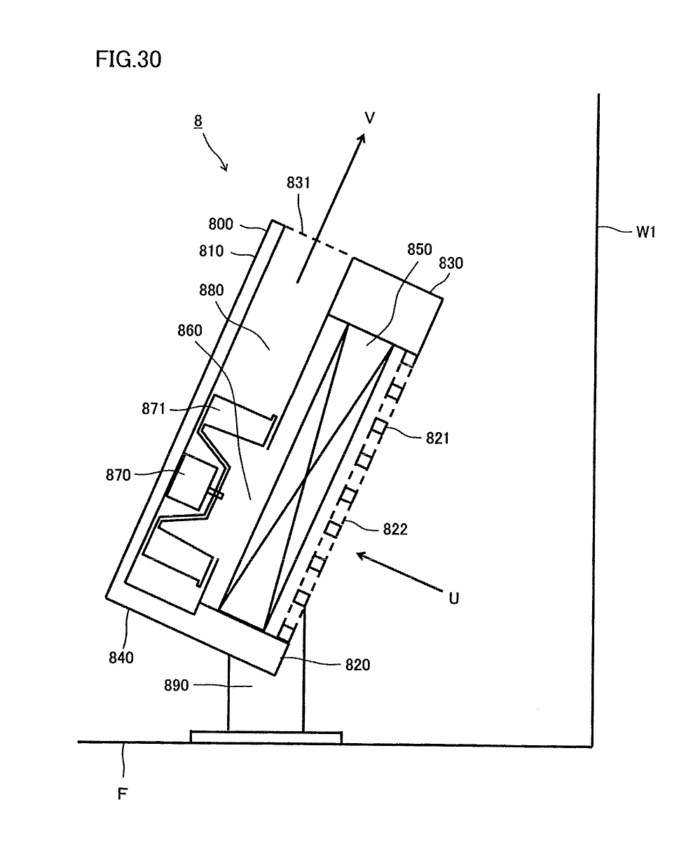

Hereinafter, an Embodiment 4-1 of the Present Invention Will be described with reference to drawings. FIG. 30 is a side cross-sectional view schematically illustrating an air cleaner of the embodiment 4-1.

As shown in FIG. 30, the air cleaner 8 comprises: a main body; a suction port 822 provided in the main body and for taking in air inside the room; an air filter 850 provided in the main body, as a removing part for removing dust and/or a substance present in the air taken in from the suction port 822; a blowout port 831 provided in the main body and for sending out the air treated by the air filter 850 into the room; and an air blower 870 provided in the main body as an air blowing part for moving the air from the suction port 822 to the blowout port 831.

The main body of the air cleaner 8 is held by a housing 800. The housing 800 is placed on a floor surface F so as to be held by a supporting part 890 and has a front surface (anterior surface) 810, a rear surface (posterior surface) 820, a top surface (upper surface) 830, and a bottom surface 840. The rear surface 820 of the housing 800 is covered by a perforated panel 821 in which a multitude of suction ports 822 are provided in a grid-like manner. Here, it is supposed that a side of the air cleaner 8, from which cleaned air is sent out in a direction indicated by an arrow V is a side of the rear surface 820 and a side opposite thereto is a side of the front surface 810, with the air cleaner 8 viewed from vertically above.

On the top surface 830 of the housing 800, the blowout port 831 is provided. The blowout port 831 is formed so as to be of a substantially rectangular shape extending in a width direction of the housing 800 and is provided, by inclining the housing 800, so as to allow the blowout port 831 to face backwardly upward.

Inside the housing 800, an air flow channel which communicates from the suction port 822 to the blowout port 831 and includes a suction flow passageway 860 and a blowout flow passageway 880 is formed. Inside the air flow channel, the air blower 870 for sending out the air is placed. As a fan constituting the air blower 870, a sirocco fan 871 is used. In the air flow channel, on a downstream side of the sirocco fan 871, the blowout flow passageway 880 for flowing the air from the sirocco fan 871 to the blowout port 831 is formed. The blowout flow passageway 880 is not inclined and is a linear flow passageway. By inclining the housing 800 toward a side wall surface W1 of the room, the blowout flow passageway 880 is allowed to guide the air sent out by the sirocco fan 871 backwardly upward. In the present embodiment, parts of the air blower 870, the sirocco fan 871, and the blowout flow passageway 880 are provided so as to be linearly arranged, and by inclining the housing 800, the air flow channel passing through the air blower 870 and the sirocco fan 871 is also inclined as similarly to the blowout flow passageway 880. The other parts are the same as those of the air cleaner 1 of the embodiment 1-1.

In the air cleaner 8 of the present embodiment, since upon driving the air cleaner 8, the housing 800 is inclined toward the side wall surface W1 of the room, it is not required to bend an air current at a position of the blowout port 831 of the air cleaner 8 in a direction toward the side wall surface W1, thereby reducing a pressure loss in the air flow channel and improving a power consumption.

Accordingly, since the blowout flow passageway 880 is not inclined, though an effect of reducing a noise is slightly inferior, an energy saving effect can be further attained, as compared with the air cleaner of the first embodiment.

In addition, since by changing a setting of an inclination angle θ of the supporting part 890, a configuration which allows an angle of an inclination of the housing 800 to be changed in accordance with a user's desire can be obtained, the inclination angle θ can be changed in accordance with a height of the room and a position in which the air cleaner is placed, thereby allowing expansion of an optimum area where the air cleaner is placed.

Furthermore, when the housing 800 is held with the inclination angle θ being set to be 0°, a depth dimension of the air cleaner 8 becomes minimum, and when the housing 800 is held with the inclination angle θ being set to be 90°, a height dimension of the air cleaner 8 becomes minimum, thereby making the air cleaner 8 compact, for example, upon storing the air cleaner and allowing drastic enhancement of convenience for a user.

### Embodiment 4-2

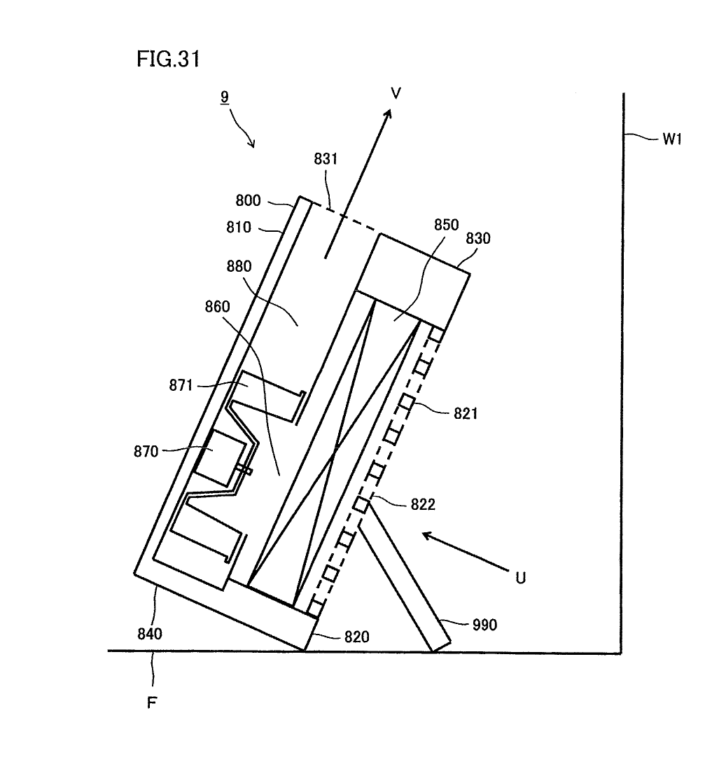

Hereinafter, an embodiment 4-2 of the present invention will be described with reference to drawings. FIG. 31 is a side cross-sectional view schematically illustrating an air cleaner of the embodiment 4-2. The same reference numerals as those used in the above descriptions of the embodiment 4-1 shown in FIG. 30 are used to denote the same parts as those in the embodiment 4-1.

As shown in FIG. 31, in the air cleaner 9 of the present embodiment, a structure of a supporting part 990 is different from that in the embodiment 4-1. The other parts are the same as those in the embodiment 4-1.

In the air cleaner 9 of the present embodiment, the supporting part 990 is provided on a side surface of a housing 800 and is set such that the housing 800 is held so as to be inclined by rotating the supporting part 990.

Accordingly, though since a part of a bottom surface 840 of the housing 800 contacts a floor surface F, air on a side of a front surface 810 of the air cleaner 9 is slightly hardly sucked, as compared with the embodiment 4-1, the substantially same effect as that attained by the air cleaner of the embodiment 4-1 can be attained.

The fourth embodiment is summed up as follows.

(1) The air cleaner is placed on the floor or the desk and used so as to be located in the vicinity of the side wall surface of the room and comprises: the main body; the suction port provided in the main body and for taking in the air inside the room; the removing part provided in the main body and for removing dust and/or a substance present in the air taken in from the suction port; the blowout port provided in the main body and for sending out the air treated by the removing part into the room; the air blowing part provided in the main body and for moving the air from the suction port to the blowout port; and the supporting part for holding the housing and keeping a posture of the housing. When the distance from the middle position of the blowout port to the side wall surface of the room is supposed to be L [mm], the distance from the middle position of the blowout port to the ceiling surface of the room is supposed to be H [mm], and the air cleaner is placed and used in the position which allows the distance L to be the value selected from among the values in the range of 100<L<600, in order to cause the air sent out from the blowout port to first reach the side wall surface of the room, the angle θ[°] formed between the direction in which the air is sent out from the blowout port and the vertically upward direction is set to be in the range of tan⁻¹(L/H)<θ≦35.

The air cleaner configured as mentioned above is characterized in that upon driving the air cleaner, the supporting part holds the housing so as to incline the housing backward and to form the angle θ set in the range of tan⁻¹(L/H)<θ≦35, with respect to the vertically upward direction.

By employing this configuration, through driving the air cleaner, the cleaned air sent out from the blowout port is sent out backwardly upward so as to form the above-mentioned angle θ with respect to the vertically upward direction. In a case where the present air cleaner is placed in the vicinity of the side wall surface of the room and the front surface of the present air cleaner is arranged so as to face a central side of the room, exhibited is a behavior of the cleaned air that the cleaned air sent out from the blowout port first reaches the side wall surface of the room, and flows upward along this side wall surface, circulates along a ceiling and the other side wall surfaces in the room, and is sucked from the suction port. When such an air current is generated, an amount of the air current sent out from the blowout port, which is immediately sucked into the air cleaner, is decreased and spreading of the cir current, in a lateral direction, which is sent out from the blowout port, is suppressed, thereby drastically extending the distance over which the air current can reach and drastically increasing the air velocity in the position in the room, which is away from the air cleaner. In addition, the air velocity in the vicinity of the wall surface of the room is also drastically increased.

As a result, an effect of improving an air environment in the position in the room, away from the air cleaner, can be enhanced and an indoor air in a wide range can be effectively cleaned. In addition, since an air current uselessly sent out can be decreased, even when an amount of an air flow volume is small, a desired effect of improving the air environment can be attained. Since the same effect of improving the air environment can be realized even with the small amount of the air flow volume, a noise is reduced. In addition, in a case where gaps are present in windows and others provided in the wall surfaces and airtightness of the room is low, the air velocity in the vicinity of the wall surface of the room can be increased, thereby attaining a positive pressure effect and allowing a reduction in an amount of dust intruding into the room from an outside of the room.

Upon driving the air cleaner, the housing is inclined toward the side wall surface of the room, it is not required to bend an air current at a position of the blowout port of the air cleaner in a direction toward the side wall surface, thereby reducing the pressure loss in the air flow channel and improving the power consumption. In addition, sound emitted from the blowout port does not directly propagate into a housing region of a user, thereby reducing the noise. Further, when the air cleaner is not being driven, it is not required to incline the housing, thereby allowing a decrease in an area where the air cleaner is placed.

(2) The air cleaner configured as mentioned above is characterized in that the supporting part holds the housing with the inclination angle θ being set to be any value in the above-mentioned range.

By employing this configuration, since by changing the setting of the inclination angle θ of the supporting part, the configuration which allows the angle of the inclination of the housing to be changed in accordance with a user's desire can be obtained, the inclination angle θ can be changed in accordance with the height of the room and the position in which the air cleaner is placed, thereby allowing the expansion of the optimum area where the air cleaner is placed.

(3) The air cleaner configured as mentioned above is characterized in that when the air cleaner is not driven, the supporting part holds the housing with the inclination angle θ being set to be 0° and/or 90°.

By employing the above-mentioned configuration, when the housing is held with the inclination angle θ being set to be 0°, a depth dimension of the air cleaner becomes minimum, and when the housing is held with the inclination angle θ being set to be 90°, a height dimension of the air cleaner becomes minimum, thereby making the air cleaner compact, for example, upon storing the air cleaner and allowing the drastic enhancement of the convenience for a user.

### Fifth Embodiment

Hereinafter, a fifth embodiment of the present invention will be described in a summarized manner.

(1) An air cleaner is characterized in that the air cleaner is placed on a floor or a desk in the vicinity of a side wall surface of a room and comprises, in a housing, a suction port for taking in air inside the room, a blowout port for sending out the air, taken in from this suction port and cleaned, into the room and arranged so as to be at a predetermined distance from the side wall surface, an air blower, and a blowout flow passageway communicating between the air blower and the blowout port; in a space upstream of the air blower and downstream of the suction port, a filter installation space is provided; in this filter installation space, an air cleaning part for collecting and removing dust and/or a substance present in the air taken in from the suction port and a humidifying part for humidifying the air taken in from the suction port are provided; the air blower is arranged such that an air blower suction port faces the filter installation space and an air blower blowout port faces upward; and the blowout port is arranged vertically above the filter installation space.

In the filter installation space, for example, an air cleaning part (for example, an air filter, an activated carbon filter, an electric dust collecting apparatus, a photocatalytic deodorizing element, etc.) for collecting and removing the dust and/or the substance contained in the air taken in from the suction port and a humidifying part for humidifying the air taken in from the suction port are provided.

In this configuration, in a dead space spreading above the filter installation space, an end portion of the blowout flow passageway and the blowout port are arranged. For example, in a case where a side cross-section of the air cleaner is a rectangular cross-section, the blowout flow passageway is not arranged in a direction of one of sides thereof but is arranged in a direction of a diagonal line thereof. Since a length of the diagonal line of the rectangular cross-section is invariably long, as compared with a length of the one of the sides thereof, a length of the blowout flow passageway is further extended in a predetermined occupied volume of the housing of the air cleaner.

Because of this, for example, by configuring the air cleaner such that a cross-sectional area of the blowout flow passageway gradually increases in accordance with an increase in proximity to a downstream part thereof, an air velocity of an air current flowing inside the blowout flow passageway is gradually reduced and kinetic energy of the air current is efficiently converted to a static pressure. In other words, a part of work done by the air blower (fan), which is a static pressure rise, is covered by the static pressure generated when the kinetic energy is converted as mentioned above, whereby energy required for the work of the air blower (fan) can be reduced and the air blower (fan) can be caused to do desired work with a low power consumption. This allows a high energy-saving air cleaner to be obtained.

(2) The air cleaner configured as mentioned above is characterized in that the blowout flow passageway is inclined toward the rear of the air cleaner. Preferably, the air cleaner configured as mentioned above is characterized in that the blowout flow passageway is curved toward the rear of the air cleaner. More preferably, the air cleaner configured as mentioned above is characterized in that the blowout port is provided on a rear surface part (posterior surface part) of the housing of the air cleaner.

In this configuration, the air blower is arranged on a front surface side of the housing of the air cleaner or a side thereof remote from the side wall surface of the room, the filter installation space is arranged in a rear surface side (posterior surface side) of the housing of the air cleaner or a side close to the side wall surface of the room, and the blowout port is arranged vertically above the filter installation space and on a rear surface side (posterior surface side) of the housing of the air cleaner or on a side close to the side wall surface of the room. In a case of the air cleaner having the above-mentioned configuration, sound emitted from the blowout port is released into the side of the rear surface of the air cleaner or the side wall surface of the room. Since sound waves has fine straight traveling properties, in order for the emitted sound to propagate into the housing region, the sound comes to be reflected by the side wall surface of the room and propagate or comes to propagate from the blowout port through diffraction. Therefore, the sound propagating directly from the blowout port into the housing region where a user is present is drastically decreased. Further, also inside the blowout flow passageway, the sound is reflected by the wall surfaces of the blowout flow passageway.

In a case where the blowout flow passageway is inclined toward the rear of the air cleaner, sound traveling straight and propagating inside the blowout flow passageway collides with a wall surface of the blowout flow passageway at least one time, one part thereof is absorbed, and the other part thereof is reflected, thereby attenuating the sound. In addition, one part of the reflected sound is attenuated through interference.

In addition, in a case where the blowout flow passageway is curved toward the rear of the air cleaner, sound traveling straight and propagating inside the blowout flow passageway collides with the wall surface of the blowout flow passageway at least one time, the one part thereof is absorbed, and the other part thereof is reflected, thereby attenuating the sound. In addition, one part of the reflected sound is attenuated through interference. Further, it does not occur that a direction of the air is abruptly changed at some position of the blowout flow passageway, thereby allowing a reduction in a pressure loss in the blowout flow passageway.

Furthermore, in a case where the blowout port is arranged in a rear surface part (posterior surface part) of the housing of the air cleaner, a diffraction angle of the sound propagating from the blowout port through the diffraction is further increased and an effect of attenuating the sound due to the diffraction is accordingly enhanced, thereby drastically attenuating the sound propagating into the housing region.

This causes the noise propagating into the housing space to be drastically attenuated, thereby allowing the housing space to be maintained quiet. With the drastically reduced noise taken into account, through increasing an air flow volume sent out from the air blower, for example, by increasing the number of rotations of the fan, dust collecting performance of the air cleaner can be drastically enhanced.

(3) The air cleaner configured as mentioned above is characterized in that the suction port is arranged on the rear surface (posterior surface) of the housing; the filter installation space is arranged on the front surface of the suction port; the air blower is arranged on the front surface of the filter installation space; the blowout port is arranged behind and above the air blower and above the filter installation space; a series of channels which are the air blower, the blowout flow passageway, and the blowout port are arranged from a bottom surface front part (front part of a lower part) of the housing to a rear part of an upper part of the housing; and the cleaned air is sent out from the blowout port backwardly upward.

In other words, the air cleaner configured as mentioned above is characterized in that the suction port is arranged on a side surface, facing the side wall surface of the room, of the air cleaner on a side from which the cleaned air is sent out, with the air cleaner viewed from vertically above; the filter installation space is arranged on a side opposite to the side wall surface of the room with respect to the suction port; the air blower is arranged on the side opposite to the side wall surface of the room with respect to the filter installation space; the blowout port is arranged above the air blower on the side of the side wall surface of the room and above the filter installation space; the series of channels which are the air blower, the blowout flow passageway, and the blowout port are arranged from a position on the bottom surface of the housing and remote from the side wall surface of the room to a position on the top surface of the housing and adjacent to the side wall surface of the room; and the cleaned air is sent out from the blowout port upward and toward the side wall surface of the room.

By employing this configuration, since the suction port is arranged on the rear surface side (posterior surface side), sound emitted from the suction port is prevented from directly leaking to the front surface side, that is, a housing space side. Before the sound reaches the housing space, a traveling direction of the sound is changed, taking a 180°-turn, thereby drastically attenuating the sound due to a diffraction effect.

This causes the noise propagating into the housing space to be drastically attenuated, thereby allowing the housing space to be maintained quiet. With the drastically reduced noise taken into account, through increasing an air flow volume sent out from the air blower, for example, by increasing the number of rotations of the fan, the dust collecting performance of the air cleaner can be drastically enhanced.

In addition, by employing this configuration, since the suction port is arranged on the rear surface (posterior surface), dust accumulated on, for example, an air filter installed in the filter installation space is hardly visible from a side of the housing space.

This greatly alleviates a problem of an aesthetic appearance. Conventionally, as a method for maintaining the aesthetic appearance of the air cleaner, a method in which the whole air filter is covered by, for example, a panel and thereby, the dust accumulated on the air filter is made hardly visible has been employed. However, in this conventional configuration, an air flow volume is drastically reduced due to a pressure loss caused by a ventilation resistance of the panel and in order to secure the same air flow volume, a noise is increased. By employing the above-mentioned configuration of the present invention, since necessity of maintaining the aesthetic appearance of the suction port is greatly alleviated, a pressure loss of the suction port can be drastically reduced, thereby allowing the noise of the air cleaner to be drastically reduced. With the drastically reduced noise taken into account, through increasing the air flow volume sent out from the air blower, for example, by increasing the number of rotations of the fan, the dust collecting performance of the air cleaner can be drastically enhanced.

Furthermore, by employing this configuration, since a length of the blowout flow passageway is further extended in a predetermined occupied volume of the housing of the air cleaner and heavy parts in the configuration, for example, such as a driving motor of the air blower, the air cleaning part, and the humidifying part are collectively contained in a lower part of the housing of the air cleaner, a center of gravity of the air cleaner is located in the lower part of the housing of the air cleaner.

This allows not only the above-mentioned effect to be obtained but also a posture of the air cleaner to be stable so as to cause the air cleaner to hardly topple down even in a case where a height of the housing of the air cleaner is high.

(4) The air cleaner configured as mentioned above is characterized in that a sound insulating part is provided further in front of the air blower and/or the blowout flow passageway.

In other words, the air cleaner configured as mentioned above is characterized in that the sound insulating part is provided on a side opposite to the side wall surface of the room with respect to the air blower and/or the blowout flow passageway.

In this configuration, in a channel through which sound emitted from the air blower and/or the blowout flow passageway, which are/is arranged in closest proximity to the housing region, propagates into the housing region, the sound insulating part is arranged, and a part of the sound emitted from the air blower and/or the blowout flow passageway is insulated by the sound insulating part.

This causes the noise propagating into the housing space to be further attenuated, thereby allowing the housing space to be maintained quiet. With the reduced noise taken into account, through increasing an air flow volume sent out from the air blower, for example, by increasing the number of rotations of the fan, the dust collecting performance of the air cleaner can be further enhanced.

(5) The air cleaner configured as mentioned above is characterized in that a sound absorbing part is provided further in front of the air blower and/or the blowout flow passageway.

In other words, the air cleaner configured as mentioned above is characterized in that the sound absorbing part is provided on a side opposite to the side wall surface of the room with respect to the air blower and/or the blowout flow passageway.

In this configuration, in a channel through which sound emitted from the air blower and/or the blowout flow passageway, which are/is arranged in closest proximity to the housing region, propagates into the housing region, the sound absorbing part is arranged, and a part of the sound emitted from the air blower and/or the blowout flow passageway is absorbed by the sound absorbing part.

This causes the noise propagating into the housing space to be further attenuated, thereby allowing the housing space to be maintained quiet. With the reduced noise taken into account, through increasing an air flow volume sent out from the air blower, for example, by increasing the number of rotations of the fan, the dust collecting performance of the air cleaner can be further enhanced.

(6) The air cleaner configured as mentioned above is characterized in that in the filter installation space, a humidifying part for humidifying the air taken in from the suction port is provided and further in front of the air blowerand/or the blowout flow passageway, a water retaining part for retaining water supplied to the humidifying part is provided.

In other words, the air cleaner configured as mentioned above is characterized in that in the filter installation space, the humidifying part for humidifying the air taken in from the suction port is provided and on a side opposite to the side wall surface of the room with respect to the air blower and/or blowout flow passageway, the water retaining part for retaining the water supplied to the humidifying part is provided.

In this configuration, in a channel through which sound emitted from the air blower and/or the blowout flow passageway, which are/is arranged in closest proximity to the housing region, propagates into the housing region, the water retaining part is arranged. This water retaining part plays a role of a sound insulating wall and brings about a sound insulating effect, whereby a part of the sound emitted from the air blower and/or the blowout flow passageway is insulated by the water retaining part.

This causes the noise propagating into the housing space to be attenuated, thereby allowing the housing space to be maintained quiet. With the reduced noise taken into account, through increasing an air flow volume sent out from the air blower, for example, by increasing the number of rotations of the fan, the dust collecting performance of the air cleaner can be enhanced.

In addition, since the water retaining part plays the role of the sound insulating part, it is not required to separately provide the sound insulating part, thereby saving a space and allowing a low-cost and quiet air cleaner to be obtained.

(7) The air cleaner configured as mentioned above is characterized in that the blowout flow passageway is inclined in the same direction as a direction in which cleaned air is sent out, with respect to a vertically upward direction and when an angle at which the blowout flow passageway is inclined, with respect to the vertically upward direction, is supposed to be the angle ψ p is set to be within a range shown in the below expression, and the blowout flow passageway is arranged inside the housing.

0<ψ≦θ

In this configuration, it does not occur that the direction of the cleaned air sent out from the air blower is abruptly changed at some position of the blowout flow passageway, the direction thereof is changed in a phased manner while the cleaned air is flowing through the blowout flow passageway, and the cleaned air is sent out from the blowout port at the angle which is set as mentioned above.

Since this allows a pressure loss in the blowout flow passageway to be reduced and an air blowing efficiency to be enhanced, the same dust collecting performance can be attained with a low power consumption and a highly energy-saving air cleaner can be obtained. In addition, the number of rotations of the fan, which is required in order to obtain the same air flow volume, can be reduced, thereby allowing a noise to be reduced and a quiet air cleaner to be obtained.

(8) The air cleaner configured as mentioned above is characterized in that a shape of a side cross-section of one of wall surfaces forming the blowout flow passageway, which is arranged in a position farthest from the side wall surface of the room, is of an arc having a central angle of the above-mentioned θ° and the side cross-section thereof is tangent to a wall surface of a casing of the air blower at a position communicating with the air blower.

In this configuration, it does not occur that a direction of the cleaned air sent out from the air blower is abruptly changed at some position of the blowout flow passageway, the direction thereof is gradually smoothly changed while the cleaned air is flowing through the blowout flow passageway, and the cleaned air is sent out from the blowout port at the angle which is set as mentioned above.

Since this allows a pressure loss in the blowout flow passageway to be reduced and an air blowing efficiency to be enhanced, the same dust collecting performance can be attained with a low power consumption, and a highly energy-saving air cleaner can be obtained. In addition, the number of rotations of the fan, which is required in order to obtain the same air flow volume, can be reduced, thereby allowing a noise to be reduced and a quiet air cleaner to be obtained.

### Comparative Embodiment

Hereinafter, a comparative embodiment according to the present invention will be described with reference to drawings.

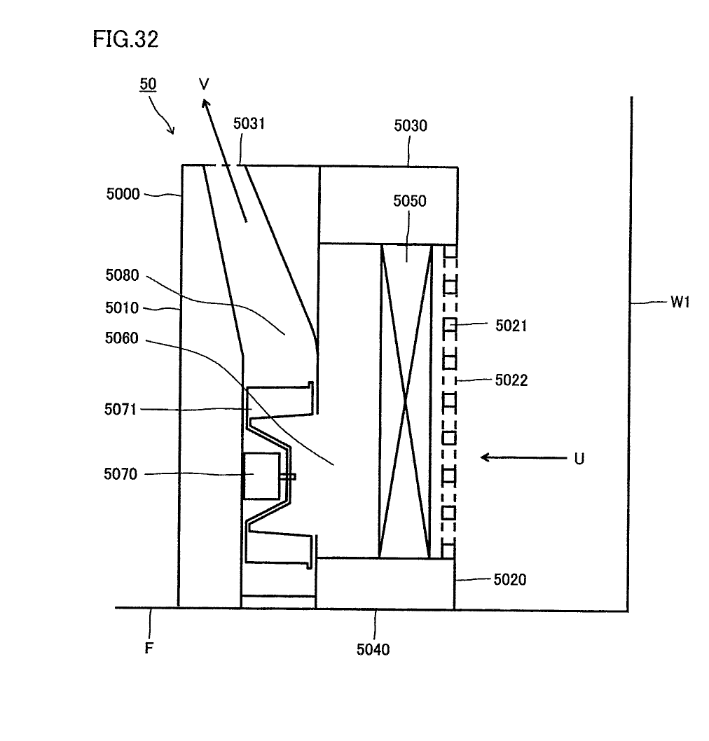

FIG. 32 is a side cross-sectional view schematically illustrating an air cleaner of the comparative embodiment according to the present invention.

As shown in FIG. 32, the air cleaner 50 is placed on a floor surface F or a desk and used so as to be located in the vicinity of a side wall surface W1 of a room. The air cleaner 50 comprises: a main body; a suction port 5022 provided in the main body and for taking in air inside the room; an air filter 5050 provided in the main body, as a removing part for removing dust and/or a substance present in the air taken in from the suction port 5022; a blowout port 5031 provided in the main body and for sending out the air treated by the air filter 5050 into the room; and an air blower 5070 provided in the main body as an air blowing part for moving the air from the suction port 5022 to the blowout port 5031.

The main body of the air cleaner 50 is held by a housing 5000. The housing 5000 is placed on the floor surface F and has a front surface (anterior surface) 5010, a rear surface (posterior surface) 5020, a top surface (upper surface) 5030, and a bottom surface 5040 contacting the floor surface F. The rear surface 5020 of the housing 5000 is covered by a perforated panel 5021 in which a multitude of suction ports 5022 are provided in a grid-like manner. Here, it is supposed that a side of the air cleaner 50, from which cleaned air is sent out in a direction indicated by an arrow V is a side of the front surface 5010 and a side opposite thereto is a side of the rear surface 5020, with the air cleaner 50 viewed from vertically above.

The housing 5000 is placed on the floor surface F so as to cause the side of the rear surface 5020 to face the side wall surface W1 of the room and to be located at a predetermined distance from the side wall surface W1. On the top surface 5030 of the housing 5000, the blowout port 5031 is provided. The blowout port 5031 is formed so as to be of a substantially rectangular shape extending in a width direction of the housing 5000 and is provided so as to face forwardly upward.

Inside the housing 5000, an air flow channel which communicates from the suction port 5022 to the blowout port 5031 and includes a suction flow passageway 5060 and a blowout flow passageway 5080 is formed. Inside the air flow channel, the air blower 5070 for sending out the air is placed. As a fan constituting the air blower 5070, a sirocco fan 5071 is used. In the air flow channel, on a downstream side of the sirocco fan 5071, the blowout flow passageway 5080 for flowing the air from the sirocco fan 5071 to the blowout port 5031 is formed. The blowout flow passageway 5080 guides the air sent out by the sirocco fan 5071 forwardly upward. A cross-sectional area of the blowout flow passageway 5080 gradually increases in accordance with an increase in proximity to the blowout port 5031 and an increase in proximity to a downstream part thereof. The blowout flow passageway 5080 has the same configuration as that shown in FIG. 2 and FIG. 3.

In the air cleaner 50 of the comparative embodiment, the blowout flow passageway 5080 is inclined toward a direction of a space (space where air is desired to be cleaned) where a person is present. The other parts are the same as those in the embodiment 1-1.

In the air cleaner 50 of the comparative embodiment, cleaned air is sent out from the blowout port 5031 toward a side of the space where the air is desired to be cleaned so as to form an angle of one value selected from among values in a range of tan⁻¹(L/H)<θ≦35 with respect to a vertically upward direction and is caused to reach a ceiling surface. Since an air current collides with the ceiling surface from an oblique direction, the air current hardly flows in a direction which forms an acute angle with respect to a wall surface with which the air current collides. In other word, since the air current easily flows in a direction (toward the side of the space where the air is desired to be cleaned) which forms an obtuse angle with respect to the wall surface with which the air current collides, an amount of the air current flowing toward the side of the space where the air is desired to be cleaned is increased, as compared with a case where the air current is sent out vertically upward (θ=0°). In other words, the air current can be sent out intentionally toward the side of the space where the air is desired to be cleaned and a distance over which the air current can reach is extended.

Accordingly, since the blowout flow passageway 5080 is inclined toward the direction of the space (space where the air is desired to be cleaned) in which a person is present, though an effect of reducing a noise is slightly inferior, the substantially same effect as that attained by the first embodiment can be attained.

Here, in a case where the cleaned air is sent out from the blowout port 5031 to the side of the space where the air is desired to be cleaned so as to form an angle, with respect to the vertically upward direction, larger than 35°, due to uncomfortable draft feeling, wind stress is likely to be imposed.

Next, a result of measuring noises by using the air cleaners of the above-described embodiment 1-1 (FIG. 1), embodiment 1-2 (FIG. 24), embodiment 3-1 (FIG. 28), embodiment 3-2 (FIG. 29), and comparative embodiment (FIG. 32) will be described in comparison with the conventional air cleaner (FIG. 34).

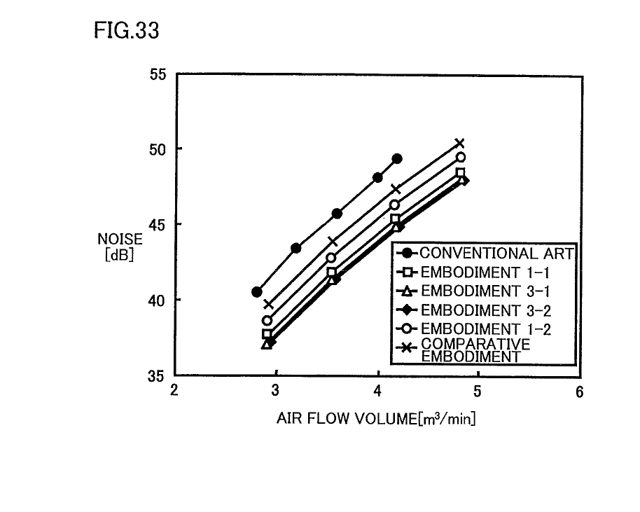

FIG. 33 is a graph showing a relationship between an air flow volume and a noise in each of the air cleaners. A horizontal axis indicates the air flow volume (unit: m³/min) of each of the air cleaners and a vertical axis indicates an average value (unit: dB) of noises in positions 1 m away from a front surface, a left side surface, a right side surface, a center of a top surface of each of the air cleaners, respectively.

First, with reference to the measurement result shown in FIG. 33, the air cleaner of the embodiment 1-1 (□ in FIG. 33) and the conventional air cleaner ( in FIG. 33) are compared.

In the air cleaner of the embodiment 1-1, the arrangement of the suction port on the rear surface side (posterior surface side) prevents sound emitted from the suction port from leaking directly to the front surface side. Before the sound reaches the front surface side, a traveling direction of the sound is changed, taking a 180°-turn, and during that time, the sound is attenuated due to a diffraction effect, thereby allowing the noise to be reduced. In addition, the arrangement of the suction port on the rear surface side (posterior surface side) solves a problem associated with an appearance, such as a problem of making dust accumulated on the filter hardly visible. In other words, since necessity of covering the whole filter with the panel is eliminated, it is made possible to drastically reduce a ventilation resistance of the panel, thereby allowing the noise to be reduced.

In addition, since the blowout flow passageway is inclined and the sirocco fan is caused to be invisible from the blowout port, viewed from vertically above, in the blowout flow passageway, sound collides with, at least one time, one of wall surfaces forming the blowout flow passageway. When the sound collides with the one of the wall surfaces, the sound is reflected. However, all of the sound is not reflected but one part thereof passes through and the other thereof is attenuated when being deprived of thermal energy upon the collision with the wall surface and of vibrational energy upon vibration with the wall surface. In addition, when the sound is reflected, interference of the sound inside the blowout flow passageway is easily caused and thereby, the sound is further attenuated, thereby allowing the noise to be reduced.

Furthermore, by inclining the blowout flow passageway in a direction opposite to a direction of the space where a person is present, the sound emitted from the blowout port more diffracts than in the conventional air cleaner and propagates through the space where a person is present, thereby enhancing an effect of attenuating the sound and allowing the noise to be reduced.

Accordingly, the air cleaner of the embodiment 1-1 allows the noise to be reduced by approximately 4 dB when an air flow volume is approximately 4 m³/min, as compared with the conventional air cleaner.

Next, the air cleaner (Δ in FIG. 33) of the embodiment 3-1 and the conventional air cleaner ( in FIG. 33) are compared.

In the air cleaner of the embodiment 3-1, the blowout port of the embodiment 1-1 is arranged so as to face the wall surface on the rear surface side (posterior surface side) of the air cleaner. In other words, in the air cleaner of the embodiment 3-1, an effect of diffracting the sound emitted from the blowout port is enhanced, as compared with the air cleaner of the embodiment 1-1, thereby allowing the noise to be reduced.

Accordingly, the air cleaner of the embodiment 3-1 allows the noise to be reduced by approximately 4.5 dB when the air flow volume is approximately 4 m³/min, as compared with the conventional air cleaner.

Next, the air cleaner (♦ in FIG. 33) of the embodiment 3-2 and the conventional air cleaner ( in FIG. 33) are compared.

In the air cleaner of the embodiment 3-2, the blowout flow passageway of the embodiment 1-1 is curved toward the direction opposite to the direction of the space where a person is present and provided such that the blowout port faces the wall surface on the rear surface side (posterior surface side) of the air cleaner. In the other words, in the air cleaner of the embodiment 3-2, since the blowout flow passageway is smoothly curved, it does not occur that a direction of the cleaned air is abruptly changed at some position of the blowout flow passageway, the direction thereof is gradually smoothly changed while the cleaned air is flowing through the blowout flow passageway, thereby allowing an increase in a flow passageway resistance in the blowout flow passageway to be more suppressed, than in the air cleaner of the embodiment 1-1. In addition, since the blowout port is provided so as to face the wall surface of the rear surface side (posterior surface side) of the air cleaner, an effect of diffracting the sound emitted from the blowout port is more enhanced than in the air cleaner of the embodiment 1-1, and the substantially same effect of reducing the noise as that attained by the air cleaner of the embodiment 3-1 can be attained.

Accordingly, the air cleaner of the embodiment 3-2 can more suppress the flow passageway resistance in the blowout flow passageway than the air cleaner of the embodiment 1-1 and the substantially same effect of reducing the noise as that attained by the air cleaner of the embodiment 3-1 can be attained, thereby allowing the noise to be reduced by approximately 4.5 dB when the air flow volume is approximately 4 m³/min, as compared with the conventional air cleaner.

Next, the air cleaner (∘ in FIG. 33) of the embodiment 1-2 and the conventional air cleaner ( in FIG. 33) are compared.

In the air cleaner of the embodiment 1-2, the parts of the air blower and the sirocco fan in the air flow channel of the embodiment 1-1 are inclined as similarly to the blowout flow passageway, thereby allowing the noise to be reduced by approximately 3 dB when the air flow volume is approximately 4 m³/min, as compared with the conventional air cleaner, though the effects due to the reflection and the interference in the blowout flow passageway are more impaired than in the embodiment 1-1.

Next, the air cleaner (X in FIG. 33) of the comparative embodiment and the conventional air cleaner ( in FIG. 33) are compared.

In the air cleaner of the comparative embodiment, the blowout flow passageway of the embodiment 1-1 is inclined toward the side of the space where a person is present, thereby allowing the noise to be reduced by approximately 2 dB when the air flow volume is approximately 4 m³/min, as compared with the conventional air cleaner, though the effect of diffracting the sound emitted from the blowout port is greatly impaired.

Finally, a method of placing the air cleaner according to the present invention will be described. The configuration of the air cleaner is the same as that of the first embodiment shown in FIG. 1 through FIG. 3. In the method of placing the air cleaner according to the present invention, as described above, the angle of the air current with respect to the vertically upward direction and the positional relationship between the air cleaner and the side wall surface of the room are extremely important.

The above-mentioned air cleaner is placed such that the angle formed by the traveling direction of the cleaned air emitted from the blowout port and the direction in which after the cleaned air emitted from the blowout port has first collided with the wall surface, the air travels along the wall surface becomes the obtuse angle. In other words, the air cleaner is placed such that the angle formed between the side wall surface and a plane including a flux of the air current, along which the cleaned air in communication with a flux (flow bundle) of the cleaned air emitted from the blowout port and a flux of the cleaned air in a position where the cleaned air emitted from the blowout port first collides with the wall surface flows, becomes an obtuse angle and such that a magnitude of the above-mentioned angle is set to be in a range of 145° through 170°, the angle being set on the side of the space (the housing space and the space where a person is present) where the air is desired to be cleaned.

In addition, the air cleaner is placed such that when a space included in the room in which the air cleaner is placed is divided into two spaces by the plane including the flux of the air current, along which the cleaned air in communication with the flux (flow bundle) of the cleaned air emitted from the blowout port and the flux of the cleaned air in the position where the cleaned air emitted from the blowout port first collides with the wall surface flows, the housing region of a user is arranged in larger one of the two divided spaces and a main traveling direction of the air current which has first collided with the wall surface is toward the larger one of the two divided spaces. In addition, the suction port is provided so as to face a smaller one of the two divided spaces.

In the above-mentioned method of placing the air cleaner, the effect of the air cleaner described in the first embodiment can be attained in an ensured manner.

Hereinbefore, although the air cleaners of the first through fifth embodiments according to the present invention are described, the present invention is not limited to the above-described embodiments but can be implemented with appropriate modifications added without departing from the scope of the present invention.

The described embodiments are to be considered in all respects only as illustrative and not restrictive. It is intended that the scope of the invention is, therefore, indicated by the appended claims rather than the foregoing descriptions of the embodiments and that all modifications and variations coming within the meaning and equivalency range of the appended claims are embraced within their scope.

### INDUSTRIAL APPLICABILITY

The present invention is applicable to an air cleaner which is placed on a floor or a desk and used so as to be located in the vicinity of a side wall surface of a room.

1. An air cleaner (1) placed on a floor or a desk and used so as to be located in a vicinity of a side wall surface of a room, comprising:
a main body (100);
a suction port (122) provided in the main body (100) and for taking in air in the room;
a removing part (150) provided in the main body (100) and for removing dust and/or a substance present in the air taken in from the suction port (122);
a blowout port (131) provided in the main body (100) and for sending out the air treated by the removing part (150) into the room; and
an air blowing part (170) provided in the main body (100) and for moving the air from the suction port (122) to the blowout port (131), wherein
when a distance from a middle position of the blowout port (131) to the side wall surface (W1) of the room is supposed to be L [mm], a distance from the middle position of the blowout port (131) to a ceiling surface (S) of the room is supposed to be H [mm], and the air cleaner (1) is placed and used in a position which allows the distance L to be a value selected from among values in a range of 100<L<600, in order to cause the air sent out from the blowout port (131) to first reach the side wall surface (W1) of the room, an angle θ[°] formed between a direction in which the air is sent out from the blowout port (131) and a vertically upward direction is set to be in a range of tan⁻¹(L/H)<θ≦35.

2. The air cleaner (1) according to claim 1, further comprising
a blowout flow passageway (180) for flowing the air from the air blowing part (170) to the blowout port (131), wherein
the blowout flow passageway (180) is arranged so as to be inclined toward the side wall surface (W1) of the room and used.

3. The air cleaner (1) according to claim 1, further comprising
a blowout flow passageway (180) for flowing the air from the air blowing part (170) to the blowout port (131),
wherein the blowout flow passageway (180) is configured so as to be inclined at an angle θ[°] fixed as one value selected from among values in a range of tan⁻¹(L/H)<θ≦35, the angle θ[°] formed between a direction of extension of a middle position of the blowout flow passageway (180) and the vertically upward direction.

4. The air cleaner (1) according to claim 3, wherein the air blowing part (170) includes a sirocco fan (171), and when a height of the sirocco fan (171) is supposed to be t, a length, in a direction along the air current, of one wall surface among wall surfaces forming the blowout flow passageway (180) is set to be t/sin θ or more, the one wall surface arranged in a position farthest from the side wall surface (W1) of the room.

5. The air cleaner (1) according to claim 2, wherein a cross-sectional area of the blowout flow passageway (180) gradually increases at a predetermined ratio in accordance with an increase in proximity to the blowout port (131).

6. The air cleaner (1) according to claim 5, wherein a width along a direction of inclination of the blowout flow passageway (180) with respect to the vertically upward direction gradually shrinks in accordance with an increase in proximity to the blowout port (131).

7. The air cleaner (3) according to claim 1, further comprising
an operation part (190) for operating the air cleaner (3), wherein
the suction port (122) is arranged on a rear surface (120) of the main body (100), the blowout port (131) is arranged on a top surface (130) of the main body (100), and the operation part (190) is arranged on a front surface (110) of the main body (100).

8. The air cleaner (1) according to claim 1, wherein the suction port (122) is arranged so as to face the side wall surface (W1) of the room and used.

9. The air cleaner (1) according to claim 1, used in a room satisfying Q/A<0.35 when an air flow volume of the air sent out from the blowout port (131) is supposed to be Q [m³/min] and an area of the floor (F) is supposed to be A [m²].
